(19) United States
(12) Patent Application Publication
(10) Pub. No.: US 2009/0221599 A1
(43) Pub. Date: Sep. 3, 2009

(54) PHTHALAZINONE PYRAZOLE DERIVATIVES, THEIR MANUFACTURE AND USE AS PHARMACEUTICAL AGENTS

(76) Inventors: Edward Boyd, Tymawr (GB); Frederick Brookfield, Benson (GB); Steve Courtney, Oxfordshire (GB); Guy Georges, Habach (DE); Bernhard Goller, Penzberg (DE); Anja Limberg, Penzberg (DE); Michael Prime, Oxon (GB); Petra Rueger, Penzberg (DE); Matthias Rueth, Penzberg (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 12/279,894

(22) PCT Filed: Mar. 16, 2007
(86) PCT No.: PCT/EP2007/002332

§ 371 Date: Oct. 7, 2008

Related U.S. Application Data
(60) Provisional application No. 60/784,134, filed on Mar. 20, 2006.

(30) Foreign Application Priority Data
Mar. 23, 2006 (EP) 06006007.6

(51) Int. Cl. A61K 31/501; C07D 403/12

(52) U.S. Cl. 514/252.05; 544/238

(57) ABSTRACT

Objects of the present invention are the compounds of formula I

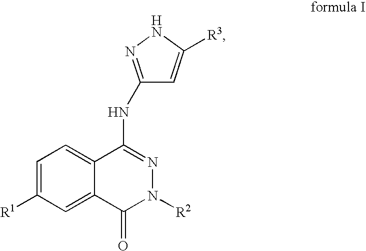

their pharmaceutically acceptable salts, enantiomeric forms, diastereoisomers and racemates, the preparation of the above-mentioned compounds, medicaments containing them and their manufacture, as well as the use of the above-mentioned compounds in the control or prevention of illnesses such as cancer.

The present invention relates to novel phthalazinone pyrazole derivatives, to a process for their manufacture, pharmaceutical compositions containing them and their manufacture as well as the use of these compounds as pharmaceutically active agents.

### BACKGROUND OF THE INVENTION

Protein kinases regulate many different signaling processes by adding phosphate groups to proteins (Hunter, T., Cell 50 (1987) 823-829); particularly serine/threonine kinases phosphorylate proteins on the alcohol moiety of serine or threonine residues. The serine/threonine kinase family includes members that control cell growth, migration, differentiation, gene expression, muscle contraction, glucose metabolism, cellular protein synthesis, and regulation of the cell cycle.

The Aurora kinases are a family of serine/threonine kinases that are believed to play a key role in the protein phosphorylation events that are essential for the completion of essential mitotic events. The Aurora kinase family is made up of three key members: Aurora A, B and C (also known as Aurora-2, Aurora-1 and Aurora-3 respectively). Aurora-1 and Aurora-2 are described in U.S. Pat. No. 6,207,401 of Sugen and in related patents and patent applications, e.g. EP 0 868 519 and EP 1 051 500.

For Aurora A there is increasing evidence that it is a novel proto-oncogene. Aurora A gene is amplified and transcript/protein is highly expressed in a majority of human tumor cell lines and primary colorectal, breast and other tumors. It has been shown that Aurora A overexpression leads to genetic instability shown by amplified centrosomes and significant increase in aneuploidy and transforms Rat1 fibroblasts and mouse NIH3T3 cells in vitro. Aurora A-transformed NIH3T3 cells grow as tumors in nude mice (Bischoff, J. R., and Plowman, G. D., Trends Cell Biol. 9 (1999) 454-459; Giet, R., and Prigent, C., J. Cell Sci. 112 (1999) 3591-3601; Nigg, E. A., Nat. Rev. Mol. Cell. Biol. 2 (2001) 21-32; Adams, R. R., et al., Trends Cell Biol. 11 (2001) 49-54). Moreover, amplification of Aurora A is associated with aneuploidy and aggressive clinical behavior (Sen, S., et al., J. Natl. Cancer Inst. 94 (2002) 1320-1329) and amplification of its locus correlates with poor prognosis for patients with node-negative breast cancer (Isola, J. J., et al., Am. J. Pathol. 147 (1995) 905-911). For these reasons it is proposed that Aurora A overexpression contributes to cancer phenotype by being involved in chromosome segregation and mitotic checkpoint control.

Human tumor cell lines depleted of Aurora A transcripts arrest in mitosis. Accordingly, the specific inhibition of Aurora kinase by selective inhibitors is recognized to stop uncontrolled proliferation, re-establish mitotic checkpoint control and lead to apoptosis of tumor cells. In a xenograft model, an Aurora inhibitor therefore slows tumor growth and induces regression (Harrington, E. A., et al., Nat. Med. 10 (2004) 262-267).

Low molecular weight inhibitors for protein kinases are widely known in the state of the art. For Aurora inhibition such inhibitors are based on i.e. quinazoline derivatives as claimed in the following patents and patent applications: WO 00/44728; WO 00/47212; WO 01/21594; WO 01/21595; WO 01/21596; WO 01/21597; WO 01/77085; WO 01/55116; WO 95/19169; WO 95/23141; WO 97/42187; WO 99/06396; pyrazole derivatives as claimed in the following patents and patent applications: WO 02/22601; WO 02/22603; WO 02/22604; WO 02/22605; WO 02/22606; WO 02/22607; WO 02/22608; WO 02/50065; WO 02/50066; WO 02/057259; WO 02/059112; WO 02/059111; WO 02/062789; WO 02/066461; WO 02/068415.

### SUMMARY OF THE INVENTION

The present invention relates to the compounds of the general formula I,

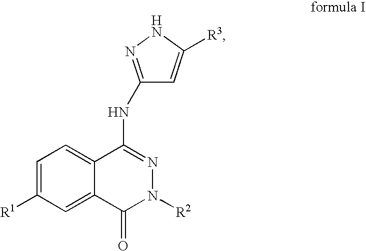

wherein
R¹ is R⁴—X— or
R⁵—X-alkylene-;
R⁴ is alkyl wherein said alkyl is substituted one to three times by hydroxy, alkoxy, carboxy, amino, alkylamino, dialkylamino, alkylsulfanyl, alkylsulfinyl, alkylsulfonyl, alkylsulfamoyl, dialkylsulfamoyl, alkylsulfonylamino, phenoxy or heterocyclylsulfonyl;
R⁵ is alkyl,
   cycloalkyl-T-,
   heterocyclyl-T-,
   aryl-T-, or
   heteroaryl-T-;
X is —S(O)₂—, —S(O)—, —C(O)NR—, —NR—, —O— or —S—;
T is a single bond or alkylene;
R² is alkyl, or
   arylalkyl, wherein the aryl is substituted one to three times by halogen;
R³ is hydrogen, alkyl or cycloalkyl;
R is hydrogen or alkyl;
and all pharmaceutically acceptable salts thereof.

The compounds according to this invention show activity as Aurora family kinase inhibitors, especially as Aurora A kinase inhibitors, and may therefore be useful for the treatment of diseases mediated by said kinase. Aurora A inhibition leads to cell cycle arrest in the G2 phase of the cell cycle and exerts an antiproliferative effect in tumor cell lines. This indicates that Aurora A inhibitors may be useful in the treatment of i.e. hyperproliferative diseases such as cancer and in particular colorectal, breast, lung, prostate, pancreatic, gastric, bladder, ovarian, melanoma, neuroblastoma, cervical, kidney or renal cancers, leukemias or lymphomas. Treatment of acute-myelogenous leukemia (AML, acute lymphocytic leukemia (ALL) and gastrointestinal stromal tumor (GIST) is included.

Objects of the present invention are the compounds of formula I and their tautomers, pharmaceutically acceptable salts, enantiomeric forms, diastereoisomers and racemates, their use as Aurora kinase inhibitors, the preparation of the above-mentioned compounds, medicaments containing them and their manufacture as well as the use of the above-mentioned compounds in treatment, control or prevention of illnesses, especially of illnesses and disorders as mentioned above like tumors or cancer (e.g. colorectal, breast, lung, prostate, pancreatic, gastric, bladder, ovarian, melanoma, neuroblastoma, cervical, kidney or renal cancers, leukemias or lymphomas) or in the manufacture of corresponding medicaments.

### DETAILED DESCRIPTION OF THE INVENTION

The term “alkyl” as used herein means a saturated, straight-chain or branched-chain hydrocarbon containing from 1 to 6, preferably 1 to 4, carbon atoms, such as methyl, ethyl, n-propyl, isopropyl, n-butyl, 2-butyl, t-butyl, n-pentyl, n-hexyl, preferably methyl, ethyl, n-propyl or isopropyl.

The term “alkoxy” as used herein means an alkyl-O-group wherein the alkyl is defined as above.

The term “alkylamino” as used herein means an alkyl-NH— group wherein the alkyl is defined as above.

The term “dialkylamino” as used herein means an (alkyl)₂N— group wherein the alkyl is defined as above.

The term “alkylene” as used herein means a saturated, straight-chain or branched-chain, preferably straight-chain hydrocarbon containing from 1 to 5, preferably from 1 to 3, carbon atoms, such as methylene, ethylene, trimethylene (1,3-propylene); tetramethylene (butylene), pentamethylene, methyl-methylene, ethyl-methylene, methyl-ethylene (1,2-propylene), ethyl-ethylene, propyl-ethylene, 1-methyl-trimethylene, 2-methyl-trimethylene, 1-ethyl-trimethylene, 2-ethyl-trimethylene and the like, preferably methylene or ethylene.

The term “halogen” as used herein means fluorine, chlorine or bromine, preferably fluorine or chlorine and more preferably fluorine.

The term “cycloalkyl” as used herein means a monocyclic saturated hydrocarbon ring with 3 to 6 ring atoms. Examples of such saturated carbocyclic groups are cyclopropyl, cyclobutyl, cyclopentyl and cyclohexyl, preferably cyclopropyl.

The term “heterocyclyl” as used herein means a saturated, monocyclic ring with 5 to 6 ring atoms which contains up to 3 heteroatoms, preferably 1 or 2 heteroatoms selected independently from N, O or S and the remaining ring atoms being carbon atoms. Preferably at least one heteroatom of the ring is nitrogen and the remaining heteroatoms are selected independently from nitrogen, oxygen or sulfur and such heterocyclyl group is preferably attached via the ring nitrogen atom. Such saturated heterocyclic group can be optionally substituted one or several times, preferably one or two times by alkyl, preferably by methyl. Preferably such saturated heterocyclic group is unsubstituted. Examples of such saturated heterocyclic groups pyrrolidinyl, morpholinyl, piperazinyl, N-methyl-piperazinyl, piperidyl, oxazolidinyl, thiazolidinyl, and the like, preferably pyrrolidinyl, morpholinyl, piperazinyl or N-methyl-piperazinyl, and more preferably morpholinyl.

The term “aryl” as used herein means a mono- or bicyclic aromatic ring with 6 to 10 ring carbon atoms. Examples of such aryl groups are phenyl and naphthyl, preferably phenyl.

The term “heteroaryl” as used herein means a mono- or bicyclic aromatic ring with 5 to 6, ring atoms, which contains up to 3, preferably 1 or 2 heteroatoms selected independently from N, O or S and the remaining ring atoms being carbon atoms. Examples of such heteroaryl groups include pyrrolyl, imidazolyl, pyrazolyl, triazolyl, tetrazolyl, furanyl, oxazolyl, isoxazolyl, thienyl, thiazolyl, pyridyl, pyrimidyl, pyridazinyl, pyrazinyl and the like, preferably pyrrolyl, imidazolyl, pyrazolyl, triazolyl, tetrazolyl, furanyl, oxazolyl, isoxazolyl, thienyl, thiazolyl, pyridyl, pyrimidyl, pyridazinyl or pyrazinyl, and more preferably pyridyl.

R¹ is R⁴—X— or R⁵—X-alkylene-.

R⁴ is alkyl wherein said alkyl is substituted one to three times, preferably one or two times by hydroxy, alkoxy, carboxy, amino, alkylamino, dialkylamino, alkylsulfanyl, alkylsulfinyl, alkylsulfonyl, alkylsulfamoyl, dialkylsulfamoyl, alkylsulfonylamino, phenoxy or heterocyclylsulfonyl, preferably by alkoxy, carboxy, dialkylamino, alkylsulfanyl, alkylsulfinyl, alkylsulfonyl, dialkylsulfamoyl, phenoxy or heterocyclylsulfonyl.

R⁵ is alkyl, cycloalkyl-T-, heterocyclyl-T-, aryl-T- or heteroaryl-T-, preferably alkyl, cycloalkyl-T-, heterocyclyl-T- or heteroaryl-T-.

X is —S(O)₂—, —S(O)—, —C(O)NR—, —NR—, —O— or —S—, preferably —S(O)₂—, —C(O)NR—, —NR—, —O— or —S—.

T is a single bond or alkylene, preferably alkylene.

R² is alkyl, or arylalkyl, wherein the aryl is substituted one to three times, preferably one or two times, by halogen, preferably by fluorine. Preferably R² is alkyl.

R³ is hydrogen, alkyl or cycloalkyl, preferably alkyl.

R is hydrogen or alkyl.

As used herein, in relation to high performance liquid chromatography the term “Tr” refers to retention time.

As used herein, in relation to mass spectrometry the term “ES+” refers to positive electrospray ionization mode.

As used herein, in relation to nuclear magnetic resonance (NMR) the term “DMSO-d6” refers to deuterated dimethylsulfoxide.

As used herein, the term “a therapeutically effective amount” of a compound means an amount of compound that is effective to prevent, alleviate or ameliorate symptoms of disease or prolong the survival of the subject being treated. Determination of a therapeutically effective amount is within the skill in the art.

The therapeutically effective amount or dosage of a compound according to this invention can vary within wide limits and may be determined in a manner known in the art. Such dosage will be adjusted to the individual requirements in each particular case including the specific compound(s) being administered, the route of administration, the condition being treated, as well as the patient being treated. In general, in the case of oral or parenteral administration to adult humans weighing approximately 70 Kg, a daily dosage of about 10 mg to about 10,000 mg, preferably from about 200 mg to about 1,000 mg, should be appropriate, although the upper limit may be exceeded when indicated. The daily dosage can be administered as a single dose or in divided doses, or for parenteral administration, it may be given as continuous infusion.

As used herein, a “pharmaceutically acceptable carrier” or a “pharmaceutically acceptable adjuvant” is intended to include any and all material compatible with pharmaceutical administration including solvents, dispersion media, coatings, antibacterial and antifungal agents, isotonic and absorption delaying agents, and other materials and compounds compatible with pharmaceutical administration. Except insofar as any conventional media or agent is incompatible with the active compound, use thereof in the compositions of the invention are contemplated. Supplementary active compounds can also be incorporated into the compositions.

The compounds of formula I can exist in different tautomeric forms and in variable mixtures thereof. All tautomeric forms of the compounds of formula I and mixtures thereof are an objective of the invention. For example, the pyrazole ring of formula I can exist in two tautomeric forms as shown here below:

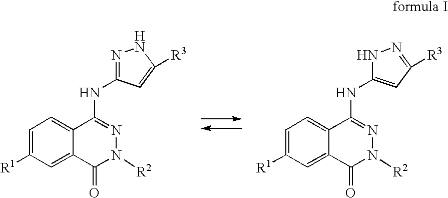

One embodiment of the invention are the compounds according to formula I, wherein
R⁴ is alkyl wherein said alkyl is substituted one or two times by alkoxy, carboxy, dialkylamino, alkylsulfanyl, alkylsulfinyl, alkylsulfonyl, dialkylsulfamoyl, phenoxy or heterocyclylsulfonyl;
R⁵ is alkyl,
   cycloalkyl-T-,
   heterocyclyl-T-, or
   heteroaryl-T-;
X is —S(O)₂—, —C(O)NR—, —NR—, —O— or —S—; and
R³ is alkyl.

Another embodiment of the invention are the compounds according to formula I, wherein
R¹ is R⁴—X—

Such compounds, for example, may be selected from the group consisting of:
2-Isopropyl-4-(5-methyl-1H-pyrazol-3-ylamino)-7-(2-methylsulfanyl-ethoxy)-2H-phthalazin-1-one;
2-Isopropyl-7-(2-methoxy-ethoxy)-4-(5-methyl-1H-pyrazol-3-ylamino)-2H-phthalazin-1-one;
2-Isopropyl-7-(2-methanesulfonyl-ethoxy)-4-(5-methyl-1H-pyrazol-3-ylamino)-2H-phthalazin-1-one;
2-Isopropyl-7-[methyl-(2-methylsulfanyl-ethyl)-amino]-4-(5-methyl-1H-pyrazol-3-ylamino)-2H-phtalazin-1-one;
4-(4-Bromo-5-methyl-1H-pyrazol-3-ylamino)-2-isopropyl-7-(2-methylsulfanyl-ethoxy)-2H-phthalazin-1-one;
2-Isopropyl-7-[(2-methoxy-ethyl)-methyl-amino]-4-(5-methyl-1H-pyrazol-3-ylamino)-2H-phthalazin-1-one;
4-(4-Bromo-5-methyl-1H-pyrazol-3-ylamino)-2-isopropyl-7-(2-methanesulfinyl-ethoxy)-2H-phthalazin-1-one;
2-Isopropyl-7-(2-methanesulfinyl-ethoxy)-4-(5-methyl-1H-pyrazol-3-ylamino)-2H-phthalazin-1-one;
2-Isopropyl-4-(5-methyl-1H-pyrazol-3-ylamino)-7-(2-methylsulfanyl-ethylsulfanyl)-2H-phthalazin-1-one;
7-[(2-Dimethylamino-ethyl)-methyl-amino ]-2-isopropyl-4-(5-methyl-1H-pyrazol-3-ylamino)-2H-phthalazin-1-one;
3-[3-Isopropyl-1-(5-methyl-1H-pyrazol-3-ylamino)-4-oxo-3,4-dihydro-phthalazin-6-yloxy]-propane-1-sulfonic acid dimethylamide;
2-Isopropyl-4-(5-methyl-1H-pyrazol-3-ylamino)-7-[3-(morpholine-4-sulfonyl)-propoxy]-2H-phthalazin-1-one;
2-Isopropyl-7-(2-methanesulfonyl-ethanesulfonyl)-4-(5-methyl-1H-pyrazol-3-ylamino)-2H-phthalazin-1-one;
7-(2-Dimethylamino-ethoxy)-2-isopropyl-4-(5-methyl-1H-pyrazol-3-ylamino)-2H-phthalazin-1-one;
7-(2-Dimethylamino-ethylamino)-2-isopropyl-4-(5-methyl-1H-pyrazol-3-ylamino)-2H-phthalazin-1-one;
4-[3-Isopropyl-1-(5-methyl-1H-pyrazol-3-ylamino)-4-oxo-3,4-dihydro-phthalazin-6-ylamino]-butyric acid;
N-[3-Isopropyl-1-(5-methyl-1H-pyrazol-3-ylamino)-4-oxo-3,4-dihydro-phthalazin-6-yl]-2-methoxy-N-methyl-acetamide;
N-[3-(3,5-Difluoro-benzyl)-1-(5-methyl-1H-pyrazol-3-ylamino)-4-oxo-3,4-dihydro-phthalazin-6-yl]-2-methoxy-N-methyl-acetamide; and
N-[3-Isopropyl-1-(5-methyl-1H-pyrazol-3-ylamino)-4-oxo-3,4-dihydro-phthalazin-6-yl]-N-methyl-2-phenoxy-acetamide.

Another embodiment of the invention are the compounds according to formula I, wherein
R¹ is R⁵—X-alkylene-.

Such compounds, for example, may be selected from the group consisting of:
7-Cyclopropylmethoxymethyl-2-isopropyl-4-(5-methyl-1H-pyrazol-3-ylamino)-2H-phthalazin-1-one;
2-Isopropyl-4-(5-methyl-1H-pyrazol-3-ylamino)-7-(pyridin-3-ylmethoxymethyl)-2H-phthalazin-1-one;
2-Isopropyl-4-(5-methyl-2H-pyrazol-3-ylamino)-7-(2-morpholin-4-yl-ethoxymethyl)-2H-phthalazin-1-one;
2-Isopropyl-4-(5-methyl-1H-pyrazol-3-ylamino)-7-methylsulfanylmethyl-2H-phthalazin-1-one; and
2-Isopropyl-7-methanesulfonylmethyl-4-(5-methyl-1H-pyrazol-3-ylamino)-2H-phthalazin-1-one.

Another embodiment of the invention are the compounds according to formula I, wherein
R² is alkyl.

Another embodiment of the invention are the compounds according to formula I, wherein
R² is alkyl;
R⁴ is alkyl wherein said alkyl is substituted one or two times by alkoxy, carboxy, dialkylamino, alkylsulfanyl, alkylsulfinyl, alkylsulfonyl, dialkylsulfamoyl, phenoxy or heterocyclylsulfonyl;
R⁵ is alkyl,
   cycloalkyl-T-,
   heterocyclyl-T-, or
   heteroaryl-T-;
X is —S(O)₂—, —C(O)NR—, —NR—, —O— or —S—; and
R³ is alkyl.

Another embodiment of the invention are the compounds according to formula I, wherein
R⁴—X—; and
R² is alkyl.

Another embodiment of the invention are the compounds according to formula I, wherein
R¹ is R⁵—X-alkylene-; and
R² is alkyl.

Another embodiment of the invention are the compounds according to formula I, wherein
R² is arylalkyl, wherein the aryl is substituted one to three times by halogen.

Another embodiment of the invention are the compounds according to formula I, wherein
R² is arylalkyl, wherein the aryl is substituted one to three times by halogen;
R⁴ is alkyl wherein said alkyl is substituted one or two times by alkoxy, carboxy, dialkylamino, alkylsulfanyl, alkylsulfinyl, alkylsulfonyl, dialkylsulfamoyl, phenoxy or heterocyclylsulfonyl;
R⁵ is alkyl,
   cycloalkyl-T-,
   heterocyclyl-T-, or
   heteroaryl-T-;
X is —S(O)₂—, —C(O)NR—, —NR—, —O— or —S—; and
R³ is alkyl.

Another embodiment of the invention are the compounds according to formula I, wherein
R¹ is R⁴—X—; and
R² is arylalkyl, wherein the aryl is substituted one to three times by halogen.

Another embodiment of the invention are the compounds according to formula I, wherein
R¹ is R⁵—X-alkylene-; and
R² is arylalkyl, wherein the aryl is substituted one to three times by halogen.

Another embodiment of the invention are the compounds according to formula I, wherein
R¹ is R⁴—X—;
R⁴ is alkyl wherein said alkyl is substituted one or two times by alkoxy, carboxy, dialkylamino, alkylsulfanyl, alkylsulfinyl, alkylsulfonyl, dialkylsulfamoyl or heterocyclylsulfonyl;
X is —NR—, —O— or —S—; and
R³ is alkyl.

Another embodiment of the invention are the compounds according to formula I, wherein
R¹ is R⁴—X—;
R⁴ is alkyl wherein said alkyl is substituted one or two times by alkoxy, alkylsulfonyl or phenoxy;
X is —S(O)₂— or —C(O)NR; and
R³ is alkyl.

Another embodiment of the invention are the compounds according to formula I, wherein
R¹ is R⁵—X-alkylene-;
R⁵ is alkyl,
   cycloalkyl-T-,
   morpholinyl-T-, or
   pyridyl-T-;
X is —S(O)₂—, —O— or —S—;
T is alkylene; and
R³ is alkyl.

Another embodiment of the invention is a process for the manufacture of the compounds of formula I, wherein
(a) the compound of formula XXVII

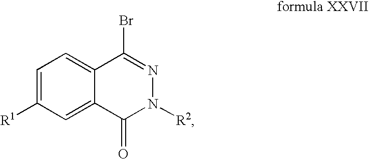

wherein R¹ and R² have the significance as given in formula I above, is reacted with a compound of formula XXVIII

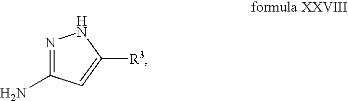

wherein R³ has the significance given in formula I above, to give the respective compound of formula I;
(b) said compound of formula I is isolated from the reaction mixture, and
(c) if desired, converted into a pharmaceutically acceptable salt.

The derivatives of the general formula I, or a pharmaceutically acceptable salt thereof, may be prepared by any process known to be applicable for the preparation of chemically-related compounds by one skilled in the art. Such processes, when used to prepare the amino pyrazole derivatives of formula I, or a pharmaceutically-acceptable salt thereof, are provided as a further feature of the invention and are illustrated by the following schemes 1 to 7 in which, unless otherwise stated, R¹, R² and R³ have the significance given herein before. Necessary starting materials may be obtained by standard procedures of organic chemistry. The preparation of such starting materials is described within the accompanying non-limiting examples. Alternatively necessary starting materials are obtainable by analogous procedures to those illustrated which are within the ordinary skill of an organic chemist.

### Scheme 1:

A preferred method for the synthesis of the derivatives of formula I, wherein R¹ is R⁴—X—, X is O and R², R³ and R⁴ are defined as above in formula I, is described in scheme 1. The derivatives of formula I, wherein R¹ is R⁴—X— and X is O, are named I-a in scheme 1.

The derivatives of formula I, wherein R¹ is R⁴—X—, X is O, and R⁴ is alkyl wherein said alkyl is substituted by sulfinyl are named I-b and the derivatives of formula I, wherein R¹ is R⁴—X—, X is O, and R⁴ is alkyl wherein said alkyl is substituted by sulfinyl are named I-b in scheme 1

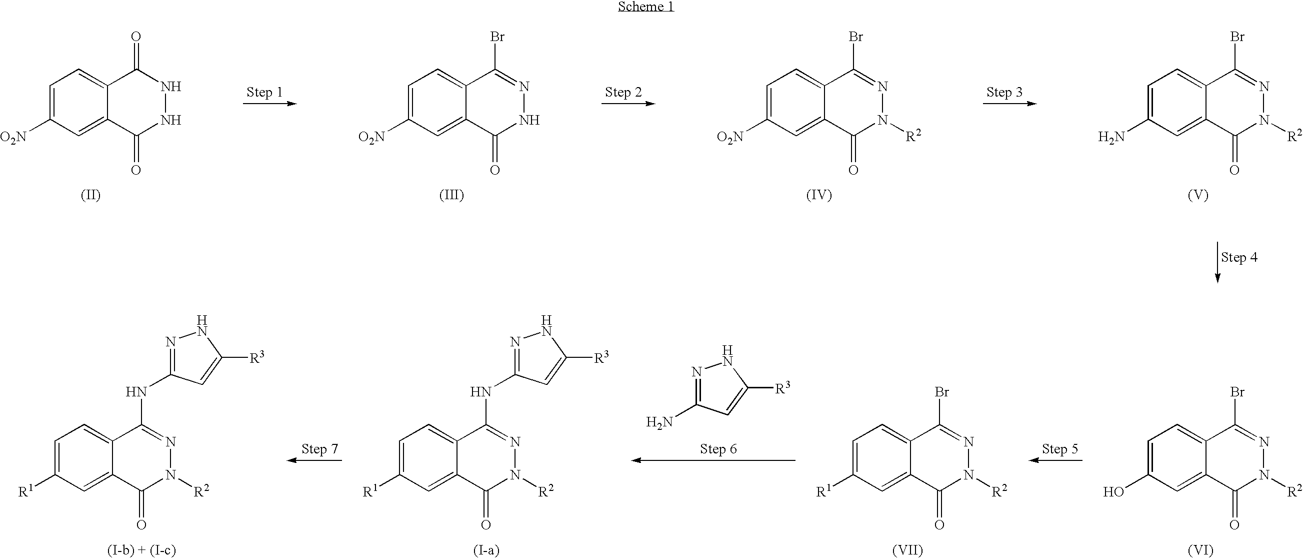

A preferred method for the synthesis of the compounds of formula I-a, I-b and I-c starts from the corresponding Phthalazine dione of formula II. Step 1 of the reaction sequence (scheme 1) is a two step process in which a dibromination is followed by a monohydrolysis, yielding the 4-bromo-nitrophthalazinone derivatives of formula III. The first step (dibromination) is typically carried out without solvent, or in solvents like dichloromethane, dichloroethane, anisole, and mixtures thereof, at temperatures between 30° C. and 150° C. Typically used brominating reagents are phosphorus oxybromide, phosphorus pentabromide and phosphorus tribromide. The second step (monohydrolysis of the dibromide) is typically carried out in aqueous or anhydrous conditions in solvents such as water, aqueous lithium hydroxide, aqueous sodium hydroxide, aqueous potassium hydroxide, aqueous sodium hydrogen carbonate, aqueous sodium carbonate, aqueous potassium hydrogen carbonate, aqueous potassium carbonate, aqueous methanol, glacial acetic acid at temperatures between 20° C. and 110° C.

In step 2, scheme 1 the obtained compounds of formula III are converted into their corresponding tertiary amides of formula IV, using methods well known to someone skilled in the art, e.g. alkylation under basic conditions. The reaction is typically carried out in aprotic solvents such as tetrahydrofuran, N,N-dimethylformamide, dimethyl sulfoxide, N-methylpyrrolidinone and mixtures thereof at temperatures between −78° C. and 100° C. Typically used bases are sodium hydride, potassium hydride, sodium methoxide, potassium tert-butoxide, lithium hexamethyldisilazide, sodium hexamethyldisilazide, potassium hexamethyldisilazide.

In step 3, scheme 1 the obtained compounds of formula IV are converted into their corresponding anilines of formula V, using methods well known to someone skilled in the art, e.g. aniline formation by the reduction of nitrobenzenes. The reaction is typically carried out in solvents like N,N-dimethylformamide, N-methylpyrrolidinone, acetonitrile, acetic acid, ethanol and methanol, and mixtures thereof, at temperatures between 20° C. and 100° C. Typically used reducing reagents are tin(II) chloride, tin(II) chloride monohydrate, iron trichloride.

In step 4, scheme 1 the obtained compounds of formula V are converted into their corresponding alcohols of formula VI, using methods well known to someone skilled in the art, e.g. diazotisation of anilines and displacement of the diazonium species with nucleophiles. The reaction is a 2 step process in which step 1 is generation of the diazonium species and step 2 is displacement of the diazonium species is carried out using a nucleophile. Step 1 of the reaction is typically carried out in solvents such as sulfuric acid, hydrochloric acid or acetic acid and mixtures thereof. Typically used reagents are sodium nitrite and isoamylnitrite with additional reagents such as urea. The first step of the reaction is typically carried out at temperatures between −10° C. and 30° C. Step 2 of the reaction is typically carried out in aqueous media such as aqueous hydrochloric acid, aqueous sulfuric acid and aqueous acetic. The second step of the reaction is typically carried out at temperatures between 20° C. and 130° C.

In step 5, scheme 1 the obtained compounds of formula VI are converted into their corresponding ethers of formula VII, using methods well known to someone skilled in the art, e.g. alkylation of phenols. The reaction is typically carried out in solvents like N,N-dimethylformamide, tetrahydrofuran, N-methylpyrrolidinone, acetonitrile, acetone, dichloromethane and dichloroethane, at temperatures between 0° C. and 100° C. Typically used bases are potassium carbonate, sodium hydride, lithium hexamethyldisilazide, sodium hexamethyldisilazide and potassium hexamethyldisilazide in conjunction with alkylating agents such as alkyl halides, alkyl mesylates and alkyl triflates.

In step 6, scheme 1 the obtained compounds of formula VII are converted into their corresponding amino pyrazoles of formula I-a, using methods well known to someone skilled in the art, e.g. palladium-mediated amination of imminobromides, vinylbromides or aryl bromides. The reaction is typically carried out in solvents such as tetrahydrofuran, toluene, alkanols such as methanol, ethanol, isopropanol, and mixtures thereof at temperatures between 40° C. and 110° C. Typically used bases are cesium carbonate, triethylamine, sodium tert-butoxide and appropriate ligated palladium (0) species can be generated using reagents such as palladium acetate, palladium dichloride, tris(dibenzylideneacetone)dipalladium, palladium tetrakis-triphenylphosphine, bis-triphenylphosphinepalladium dichloride in conjunction with phosphine based ligands such as 2,2′-bi(phenylphosphino)-1,1′-binaphthyl, 4,5-Bis(diphenylphosphino)-9,9 dimethylxanthene and 2-(di-tert-butylphosphino)biphenyl.

In step 7, scheme 1, where the R⁴ groups contain a sulfanyl group, the obtained compounds of formula I-a are converted into their corresponding sulfoxides and sulfones of formula I-b and I-c using methods well known to someone skilled in the art, e.g. oxidation of thioethers to sulfoxides and sulfones. The reaction is typically carried out in solvents such as tetrahydrofuran, toluene, alkanols such as methanol, ethanol, isopropanol and water and mixtures thereof at temperatures between 0° C. and 110° C. Typically used reagents are OXONE™ and meta-chloroperbenzoic acid.

### Scheme 2:

A preferred method for the synthesis of the derivatives of formula I, wherein R¹ is R⁴—X—, X is —N(alkyl)- and R², R³ and R⁴ are defined as above in formula I, is described in scheme 2. The derivatives of formula I, wherein R¹ is R⁴—X— and X is —N(alkyl)-, are named I-d in scheme 21.

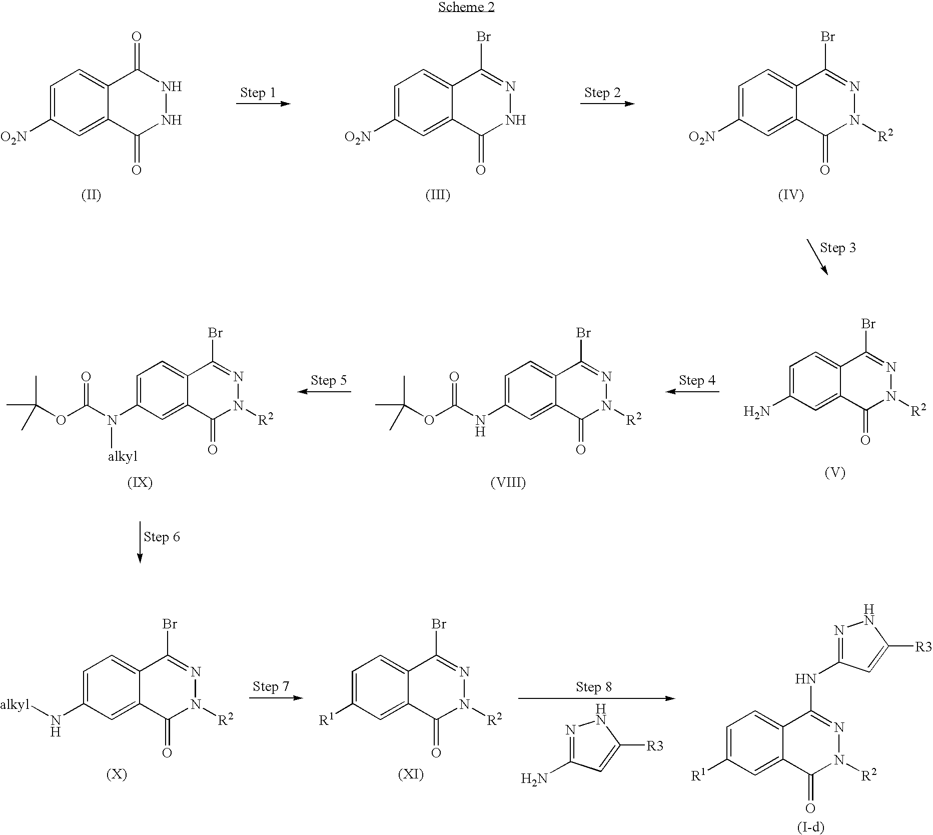

A preferred method for the synthesis of the compounds of formula Id starts from the corresponding Phthalazine dione of formula II. Step 1 of the reaction sequence (scheme 2) is a two step process in which a dibromination is followed by a monohydrolysis, yielding the 4-bromo-nitrophthalazinone derivatives of formula III. The first step (dibromination) is typically carried out without solvent, or in solvents like dichloromethane, dichloroethane, anisole, and mixtures thereof, at temperatures between 30° C. and 150° C. Typically used brominating reagents are phosphorus oxybromide, phosphorus pentabromide and phosphorus tribromide. The second step (monohydrolysis of the dibromide) is typically carried out in aqueous or anhydrous conditions in solvents such as water, aqueous lithium hydroxide, aqueous sodium hydroxide, aqueous potassium hydroxide, aqueous sodium hydrogen carbonate, aqueous sodium carbonate, aqueous potassium hydrogen carbonate, aqueous potassium carbonate, aqueous methanol, glacial acetic acid at temperatures between 20° C. and 110° C.

In step 2, scheme 2 the obtained compounds of formula III are converted into their corresponding tertiary amides of formula IV, using methods well known to someone skilled in the art, e.g. alkylation under basic conditions. The reaction is typically carried out in aprotic solvents such as tetrahydrofuran, N,N-dimethylformamide, dimethyl sulfoxide, N-methylpyrrolidinone and mixtures thereof at temperatures between −78° C. and 100° C. Typically used bases are sodium hydride, potassium hydride, sodium methoxide, potassium tert-butoxide, lithium hexamethyldisilazide, sodium hexamethyldisilazide, potassium hexamethyldisilazide.

In step 3, scheme 2 the obtained compounds of formula IV are converted into their corresponding anilines of formula V, using methods well known to someone skilled in the art, e.g. aniline formation by the reduction of nitrobenzenes. The reaction is typically carried out in solvents like N,N-dimethylformamide, N-methylpyrrolidinone, acetonitrile, acetic acid, ethanol and methanol, and mixtures thereof, at temperatures between 20° C. and 100° C. Typically used reducing reagents are tin(II) chloride, tin(II) chloride monohydrate, iron trichloride.

In step 4, scheme 2 the obtained compounds of formula V are converted into their corresponding secondary carbamates of formula VIII, using methods well known to someone skilled in the art, e.g. tert-butyloxycarbonylation of amines. The reaction is typically carried out in solvents like N,N-dimethylformamide, N-methylpyrrolidinone, acetonitrile, dichloromethane and dichloroethane, at temperatures between 0° C. and 100° C. Typically used bases are imidazole, triethylamine, N,N-diisopropylethylamine, N,N-dimethylaminopyridine and sodium hydride in conjunction with reagents such as di-tert-butyl dicarbonate.

In step 5, scheme 2 the obtained compounds of formula VIII are converted into their corresponding tertiary carbamates of formula IX, using methods well known to someone skilled in the art, e.g. alkylation of secondary carbamates. The reaction is typically carried out in solvents like N,N-dimethylformamide, N-methylpyrrolidinone, acetonitrile, acetone, dichloromethane and dichloroethane, at temperatures between 0° C. and 100° C. Typically used bases are potassium carbonate, sodium hydride, lithium hexamethyldisilazide, sodium hexamethyldisilazide and potassium hexamethyldisilazide in conjunction with alkylating agents such as alkyl halides, alkyl mesylates and alkyl triflates.

In step 6, scheme 2 the obtained compounds of formula IX are converted into their corresponding secondary amines of formula X, using methods well known to someone skilled in the art, e.g. deprotection of acid labile protecting groups such as a tert-butyloxycarbonyl group. The reaction is typically carried out without solvent or in solvents like diethyl ether, dioxane, tetrahydrofuran, dichloromethane and dichloroethane or mixtures thereof, at temperatures between 0° C. and 40° C. Typically used acids are trifluoroacetic acid, trifluoromethane sulfonic acid, aqueous hydrochloric acid, aqueous sulfuric acid or anhydrous hydrogen chloride.

In step 7, scheme 2 the obtained compounds of formula X are converted into their corresponding tertiary amines of formula XI, using methods well known to someone skilled in the art, e.g. alkylation of secondary amines. The reaction is typically carried out in solvents like N,N-dimethylformamide, N-methylpyrrolidinone, acetonitrile, acetone, dichloromethane and dichloroethane, at temperatures between 0° C. and 100° C. Typically used bases are potassium carbonate, sodium hydride, lithium hexamethyldisilazide, sodium hexamethyldisilazide and potassium hexamethyldisilazide in conjunction with alkylating agents such as alkyl halides, alkyl mesylates and alkyl triflates.

In step 8, scheme 2 the obtained compounds of formula XI are converted into their corresponding amino pyrazoles of formula I-d, using methods well known to someone skilled in the art, e.g. palladium-mediated amination of imminobromides, vinylbromides or aryl bromides. The reaction is typically carried out in solvents such as tetrahydrofuran, toluene, alkanols such as methanol, ethanol, isopropanol, and mixtures thereof at temperatures between 40° C. and 110° C. Typically used bases are cesium carbonate, triethylamine, sodium tert-butoxide and appropriate ligated palladium (0) species can be generated using reagents such as palladium acetate, palladium dichloride, tris(dibenzylideneacetone)dipalladium, palladium tetrakis-triphenylphospine, bis-triphenylphosphinepalladium dichloride in conjunction with phosphine based ligands such as 2,2′-bi(phenylphosphino)-1,1′-binaphthyl, 4,5-Bis(diphenylphosphino)-9,9 dimethylxanthene and 2-(di-tert-butylphosphino)biphenyl.

### Scheme 3:

A preferred method for the synthesis of the derivatives of formula I, wherein R¹ is R⁴—X—, X is —S—, or —S(O)₂— and R², R³ and R⁴ are defined as above in formula I, is described in scheme 3. The derivatives of formula I, wherein R¹ is R⁴—X— and X is —S—are named I-e and the derivatives of formula I, wherein R¹ is R⁴—X— and X is —S(O)₂—are named I-f in scheme 3.

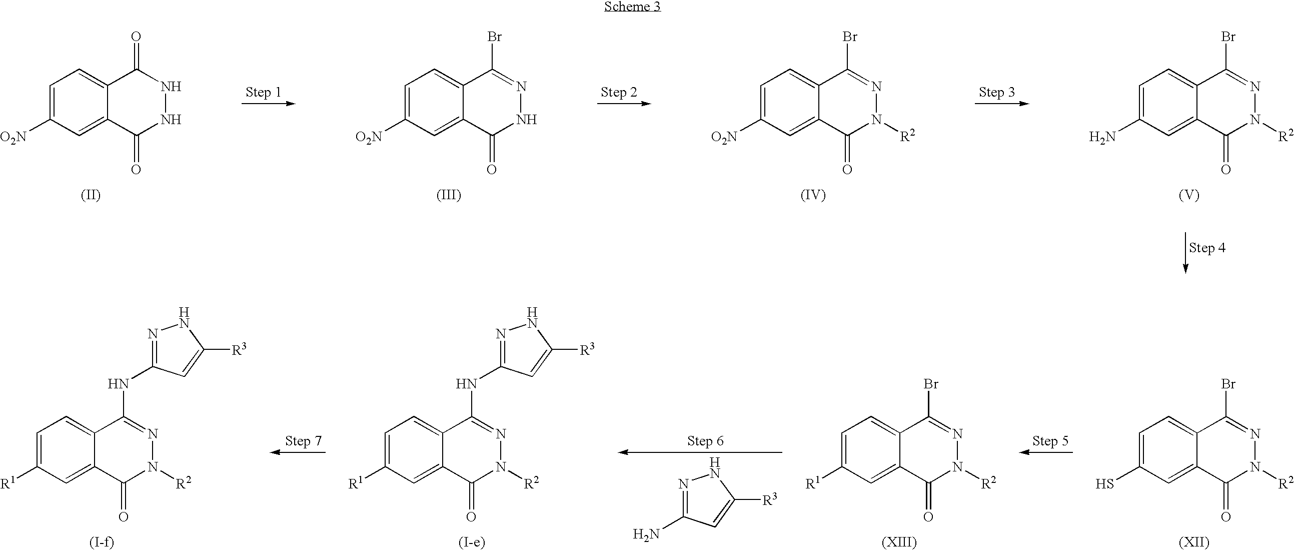

A preferred method for the synthesis of the compounds of formula I-e and I-f starts from the corresponding Phthalazine dione of formula II. Step 1 of the reaction sequence (scheme 3) is a two step process in which a dibromination is followed by a monohydrolysis, yielding the 4-bromo-nitrophthalazinone derivatives of formula III. The first step (dibromination) is typically carried out without solvent, or in solvents like dichloromethane, dichloroethane, anisole, and mixtures thereof, at temperatures between 30° C. and 150° C. Typically used brominating reagents are phosphorus oxybromide, phosphorus pentabromide and phosphorus tribromide. The second step (monohydrolysis of the dibromide) is typically carried out in aqueous or anhydrous conditions in solvents such as water, aqueous lithium hydroxide, aqueous sodium hydroxide, aqueous potassium hydroxide, aqueous sodium hydrogen carbonate, aqueous sodium carbonate, aqueous potassium hydrogen carbonate, aqueous potassium carbonate, aqueous methanol, glacial acetic acid at temperatures between 20° C. and 110° C.

In step 2, scheme 3 the obtained compounds of formula III are converted into their corresponding tertiary amides of formula IV, using methods well known to someone skilled in the art, e.g. alkylation under basic conditions. The reaction is typically carried out in aprotic solvents such as tetrahydrofuran, N,N-dimethylformamide, dimethyl sulfoxide, N-methylpyrrolidinone and mixtures thereof at temperatures between −78° C. and 100° C. Typically used bases are sodium hydride, potassium hydride, sodium methoxide, potassium tert-butoxide, lithium hexamethyldisilazide, sodium hexamethyldisilazide, potassium hexamethyldisilazide.

In step 3, scheme 3 the obtained compounds of formula IV are converted into their corresponding anilines of formula V, using methods well known to someone skilled in the art, e.g. aniline formation by the reduction of nitrobenzenes. The reaction is typically carried out in solvents like N,N-dimethylformamide, N-methylpyrrolidinone, acetonitrile, acetic acid, ethanol and methanol, and mixtures thereof, at temperatures between 20° C. and 100° C. Typically used reducing reagents are tin(II) chloride, tin(II) chloride monohydrate, iron trichloride.

In step 4, scheme 3 the obtained compounds of formula V are converted into their corresponding thiols of formula XII, using methods well known to someone skilled in the art, e.g. diazotisation of anilines and displacement of the diazonium species with nucleophiles. The reaction is a 2 step process in which step 1 is generation of the diazonium species and step 2 is displacement of the diazonium species using a nucleophile. Step 1 of the reaction is typically carried out in solvents such as sulfuric acid, hydrochloric acid or acetic acid and mixtures thereof. Typically used reagents are sodium nitrite and isoamylnitrite with additional reagents such as urea. The first step of the reaction is typically carried out at temperatures between −10° C. and 30° C. Step 2 of the reaction is typically carried out in aqueous media such as aqueous hydrochloric acid, aqueous sulfuric acid and aqueous acetic. Step 2 of the reaction is typically carried out in solvents such as tetrahydrofuran. Typically used reagents are potassium ethyl xanthate, followed by sodium hydroxide. The second step of the reaction is typically carried out at temperatures between 20° C. and 120° C.

In step 5, scheme 3 the obtained compounds of formula XII are converted into their corresponding ethers of formula XIII, using methods well known to someone skilled in the art, e.g. alkylation of thiophenols. The reaction is typically carried out in solvents like N,N-dimethylformamide, tetrahydrofuran, N-methylpyrrolidinone, acetonitrile, acetone, dichloromethane and dichloroethane, at temperatures between 0° C. and 100° C. Typically used bases are potassium carbonate, sodium hydride, lithium hexamethyldisilazide, sodium hexamethyldisilazide and potassium hexamethyldisilazide in conjunction with alkylating agents such as alkyl halides, alkyl mesylates and alkyl triflates.

In step 6, scheme 3 the obtained compounds of formula XIII are converted into their corresponding amino pyrazoles of formula I-e, using methods well known to someone skilled in the art, e.g. palladium-mediated amination of imminobromides, vinylbromides or aryl bromides. The reaction is typically carried out in solvents such as tetrahydrofuran, toluene, alkanols such as methanol, ethanol, isopropanol, and mixtures thereof at temperatures between 40° C. and 110° C. Typically used bases are cesium carbonate, triethylamine, sodium tert-butoxide and appropriate ligated palladium (0) species can be generated using reagents such as palladium acetate, palladium dichloride, tris(dibenzylideneacetone)dipalladium, palladium tetrakis-triphenylphospine, bis-triphenylphosphinepalladium dichloride in conjunction with phosphine based ligands such as 2,2′-bi(phenylphosphino)-1,1′-binaphthyl, 4,5-Bis(diphenylphosphino)-9,9 dimethylxanthene and 2-(di-tert-butylphosphino)biphenyl.

In step 7, scheme 3, where X═S, the obtained compounds of formula I-e are converted into their corresponding sulfones of formula I-f, using methods well known to someone skilled in the art, e.g. oxidation of thioethers to sulfoxides or sulfones. The reaction is typically carried out in solvents such as tetrahydrofuran, toluene, alkanols such as methanol, ethanol, isopropanol and water and mixtures thereof at temperatures between 0° C. and 110° C. Typically used reagents are OXONE™ and meta-chloroperbenzoic acid.

### Scheme 4:

A preferred method for the synthesis of the derivatives of formula I, wherein R¹ is R⁴—X—, X is —NH— and R², R³ and R⁴ are defined as above in formula I, is described in scheme 4. The derivatives of formula I, wherein R¹ is R⁴—X— and X is —NH— are named I-g in scheme 4.

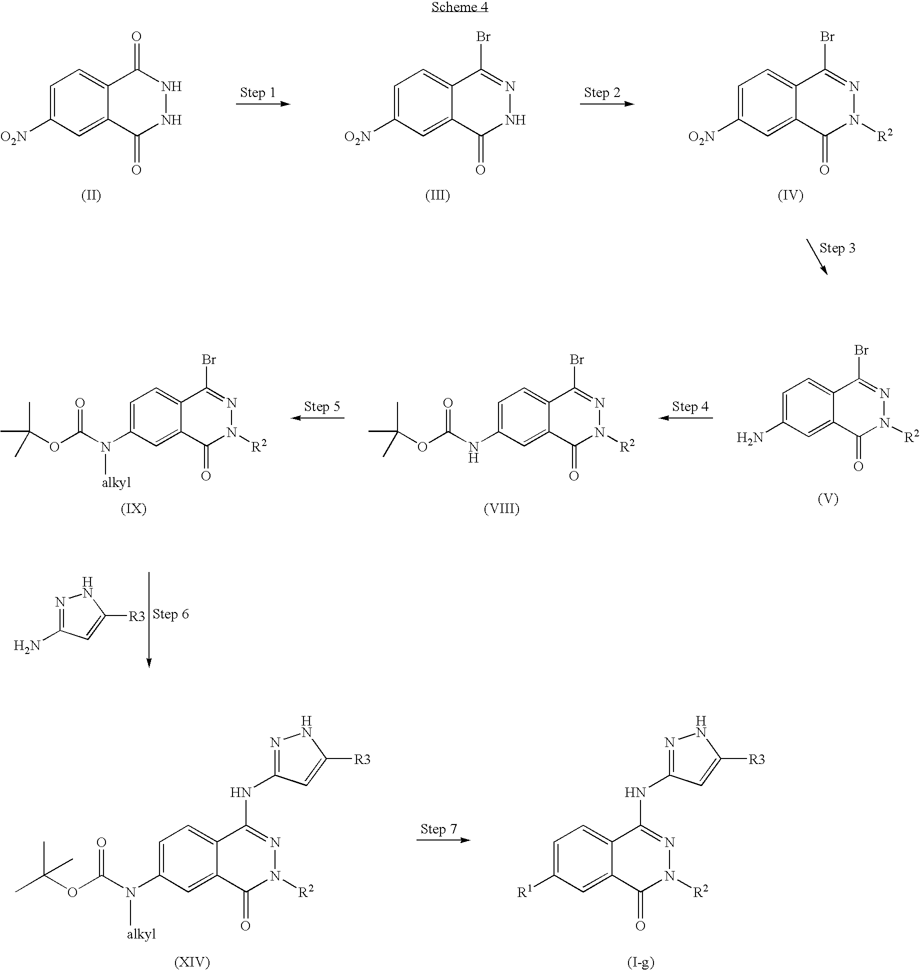

A preferred method for the synthesis of the compounds of formula I-g starts from the corresponding Phthalazine diones of formula II. Step 1 of the reaction sequence (scheme 4) is a two step process in which a dibromination is followed by a monohydrolysis, yielding the 4-bromo-nitrophthalazinone derivatives of formula III. The first step (dibromination) is typically carried out without solvent, or in solvents like dichloromethane, dichloroethane, anisole, and mixtures thereof, at temperatures between 30° C. and 150° C. Typically used brominating reagents are phosphorus oxybromide, phosphorus pentabromide and phosphorus tribromide. The second step (monohydrolysis of the dibromide) is typically carried out in aqueous or anhydrous conditions in solvents such as water, aqueous lithium hydroxide, aqueous sodium hydroxide, aqueous potassium hydroxide, aqueous sodium hydrogen carbonate, aqueous sodium carbonate, aqueous potassium hydrogen carbonate, aqueous potassium carbonate, aqueous methanol, glacial acetic acid at temperatures between 20° C. and 110° C.

In step 2, scheme 4 the obtained compounds of formula III are converted into their corresponding tertiary amides of formula IV, using methods well known to someone skilled in the art, e.g. alkylation under basic conditions. The reaction is typically carried out in aprotic solvents such as tetrahydrofuran, N,N-dimethylformamide, dimethyl sulfoxide, N-methylpyrrolidinone and mixtures thereof at temperatures between −78° C. and 100° C. Typically used bases are sodium hydride, potassium hydride, sodium methoxide, potassium tert-butoxide, lithium hexamethyldisilazide, sodium hexamethyldisilazide, potassium hexamethyldisilazide.

In step 3, scheme 4 the obtained compounds of formula IV are converted into their corresponding anilines of formula V, using methods well known to someone skilled in the art, e.g. aniline formation by the reduction of nitrobenzenes. The reaction is typically carried out in solvents like N,N-dimethylformamide, N-methylpyrrolidinone, acetonitrile, acetic acid, ethanol and methanol, and mixtures thereof, at temperatures between 20° C. and 100° C. Typically used reducing reagents are tin(II) chloride, tin(II) chloride monohydrate, iron trichloride.

In step 4, scheme 4 the obtained compounds of formula V are converted into their corresponding secondary carbamates of formula VIII, using methods well known to someone skilled in the art, e.g. tert-butyloxycarbonylation of amines. The reaction is typically carried out in solvents like N,N-dimethylformamide, N-methylpyrrolidinone, acetonitrile, dichloromethane and dichloroethane, at temperatures between 0° C. and 100° C. Typically used bases are imidazole, triethylamine, N,N-diisopropylethylamine, N,N-dimethylaminopyridine and sodium hydride in conjunction with reagents such as di-tert-butyl dicarbonate.

In step 5, scheme 4 the obtained compounds of formula VIII are converted into their corresponding tertiary carbamates of formula IX, using methods well known to someone skilled in the art, e.g. alkylation of secondary carbamates. The reaction is typically carried out in solvents like N,N-dimethylformamide, N-methylpyrrolidinone, acetonitrile, acetone, dichloromethane and dichloroethane, at temperatures between 0° C. and 100° C. Typically used bases are potassium carbonate, sodium hydride, lithium hexamethyldisilazide, sodium hexamethyldisilazide and potassium hexamethyldisilazide in conjunction with alkylating agents such as alkyl halides, alkyl mesylates and alkyl triflates.

In step 6, scheme 4 the obtained compounds of formula IX are converted into their corresponding amino pyrazoles of formula XIV, using methods well known to someone skilled in the art, e.g. palladium-mediated amination of imminobromides, vinylbromides or aryl bromides. The reaction is typically carried out in solvents such as tetrahydrofuran, toluene, alkanols such as methanol, ethanol, isopropanol, and mixtures thereof at temperatures between 40° C. and 110° C. Typically used bases are cesium carbonate, triethylamine, sodium tert-butoxide and appropriate ligated palladium (0) species can be generated using reagents such as palladium acetate, palladium dichloride, tris(dibenzylideneacetone)dipalladium, palladium tetrakis-triphenylphospine, bis-triphenylphosphinepalladium dichloride in conjunction with phosphine based ligands such as 2,2′-bi(phenylphosphino)-1,1′-binaphthyl, 4,5-Bis(diphenylphosphino)-9,9 dimethylxanthene and 2-(di-tert-butylphosphino)biphenyl.

In step 7, scheme 4 the obtained compounds of formula XIV are converted into their corresponding secondary amines of formula I-g, using methods well known to someone skilled in the art, e.g. deprotection of acid labile protecting groups such as a tert-butyloxycarbonyl group. The reaction is typically carried out without solvent or in solvents like diethyl ether, dioxane, tetrahydrofuran, dichloromethane and dichloroethane or mixtures thereof, at temperatures between 0° C. and 40° C. Typically used acids are trifluoroacetic acid, trifluoromethane sulfonic acid, aqueous hydrochloric acid, aqueous sulfuric acid or anhydrous hydrogen chloride.

### Scheme 5:

A preferred method for the synthesis of the derivatives of formula I, wherein R¹ is R⁵—X-alkylene-, X is —O— and R², R³ and R⁵ are defined as above in formula I, is described in scheme 5. The derivatives of formula I, wherein R¹ is wherein R¹ is R⁵—X-alkylene- and X is —O— are named I-h in scheme 5.

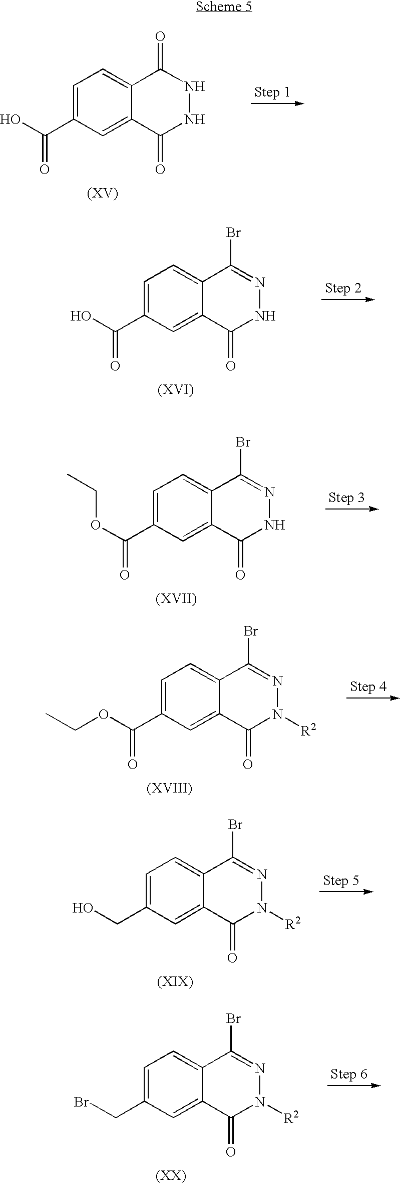

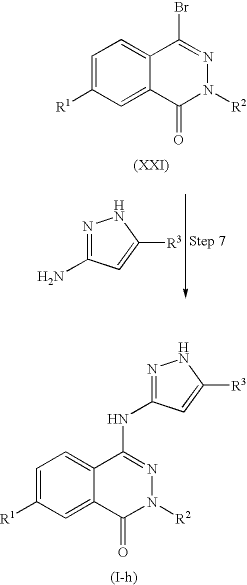

A preferred method for the synthesis of the compounds of formula I-h starts from the Phthalazine dione of formula XV. Step 1 of the reaction sequence (scheme 5) is a two step process in which a dibromination is followed by a monohydrolysis, yielding the 4-bromo-alkylcarboxyphthalazinone derivatives of formula XVI. The first step (dibromination) is typically carried out without solvent, or in solvents like dichloromethane, dichloroethane, anisole, and mixtures thereof, at temperatures between 30° C. and 150° C. Typically used brominating reagents are phosphorus oxybromide, phosphorus pentabromide and phosphorus tribromide. The second step (monohydrolysis of the dibromide) is typically carried out in aqueous or anhydrous conditions in solvents such as water, aqueous lithium hydroxide, aqueous sodium hydroxide, aqueous potassium hydroxide, aqueous sodium hydrogen carbonate, aqueous sodium carbonate, aqueous potassium hydrogen carbonate, aqueous potassium carbonate, aqueous methanol, glacial acetic acid at temperatures between 20° C. and 100° C.

In step 2, scheme 5 the obtained compounds of formula XVI are converted into their corresponding esters of formula XVII, using methods well known to someone skilled in the art, e.g. esterification under acidic conditions. The reaction is typically carried out in protic solvents such as ethanol at temperatures between 20° C. and 100° C. Typically used acids are aqueous sulfuric acid, aqueous hydrochloric acid and aqueous acetic acid.

In step 3, scheme 5 the obtained compounds of formula XVII are converted into their corresponding tertiary amides of formula XVIII, using methods well known to someone skilled in the art, e.g. alkylation under basic conditions. The reaction is typically carried out in aprotic solvents such as tetrahydrofuran, N,N-dimethylformamide, dimethyl sulfoxide, N-methylpyrrolidinone and mixtures thereof at temperatures between −78° C. and 100° C. Typically used bases are sodium hydride, potassium hydride, sodium methoxide, potassium tert-butoxide, lithium hexamethyldisilazide, sodium hexamethyldisilazide, potassium hexamethyldisilazide.

In step 4, scheme 5 the obtained compounds of formula XVIII are converted into their corresponding alcohols of formula XIX, using methods well known to someone skilled in the art, e.g. reduction of esters to form alcohols. The reaction is typically carried out in aprotic solvents like tetrahydrofuran, dioxane, dichloromethane and mixtures thereof, or, in protic solvents such as methanol. The reaction is typically carried out at temperatures between 0° C. and 100° C. Typically used reducing reagents are lithium borohydride.

In step 5, scheme 5 the obtained compounds of formula XIX are converted into their corresponding alkyl bromides of formula XX, using methods well known to someone skilled in the art, e.g. functional group interconversion of alcohols into bromides. The reaction is typically carried out in solvents like acetonitrile, tetrahydrofuran, dioxane, dichloromethane and mixtures thereof, at temperatures between 0° C. and 100° C. Typically used brominating reagents are trimethylsilyl chloride or trimethylsilyl bromide in conjunction with lithium bromide.

In step 6, scheme 5 the obtained compounds of formula XX are converted into their corresponding ethers of formula XXI, using methods well known to someone skilled in the art, e.g. alkylation of alcohols. The reaction is typically carried out in solvents like N,N-dimethylformamide, tetrahydrofuran, N-methylpyrrolidinone, acetonitrile, acetone, dichloromethane and dichloroethane, at temperatures between 0° C. and 100° C. Typically used bases are potassium carbonate, sodium hydride, lithium hexamethyldisilazide, sodium hexamethyldisilazide and potassium hexamethyldisilazide in conjunction with nucleophiles such as alcohols.

In step 7, scheme 5 the obtained compounds of formula XXI are converted into their corresponding amino pyrazoles of formula I-h, using methods well known to someone skilled in the art, e.g. palladium-mediated amination of imminobromides, vinylbromides or aryl bromides. The reaction is typically carried out in solvents such as tetrahydrofuran, toluene, alkanols such as methanol, ethanol, isopropanol, and mixtures thereof at temperatures between 40° C. and 110° C. Typically used bases are cesium carbonate, triethylamine, sodium tert-butoxide and appropriate ligated palladium (0) species can be generated using reagents such as palladium acetate, palladium dichloride, tris(dibenzylideneacetone)dipalladium, palladium tetrakis-triphenylphospine, bis-triphenylphosphinepalladium dichloride in conjunction with phosphine based ligands such as 2,2′-bi(phenylphosphino)-1,1′-binaphthyl, 4,5-Bis(diphenylphosphino)-9,9 dimethylxanthene and 2-(di-tert-butylphosphino)biphenyl.

### Scheme 6:

A preferred method for the synthesis of the derivatives of formula I, wherein R¹ is R⁵—X-alkylene-, X is —S—, or —S(O)₂— and R², R³ and R⁵ are defined as above in formula I, is described in scheme 6. The derivatives of formula I, wherein R¹ is wherein R¹ is R⁵—X-alkylene- and X is —S— are named I-i and the derivatives of formula I, wherein R¹ is wherein R¹ is R⁵—X-alkylene- and X is —S(O)₂— are named I-j in scheme 6.

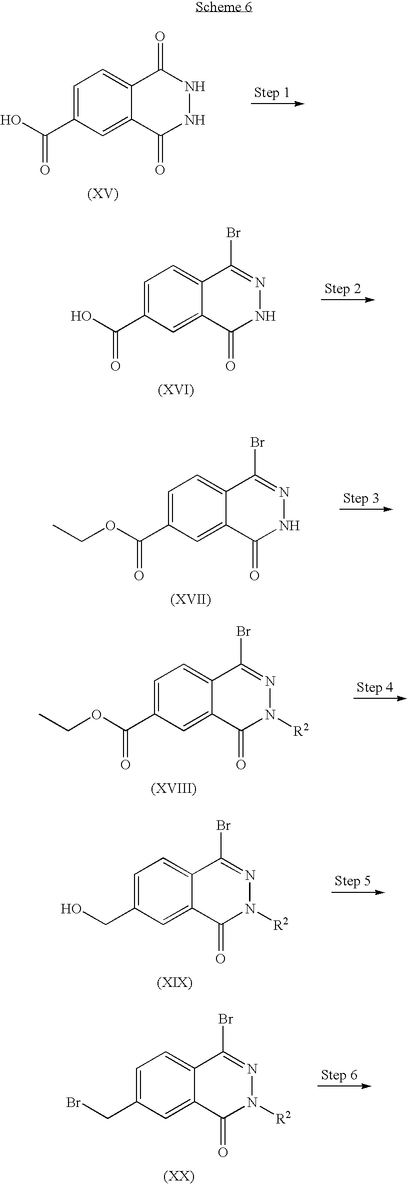

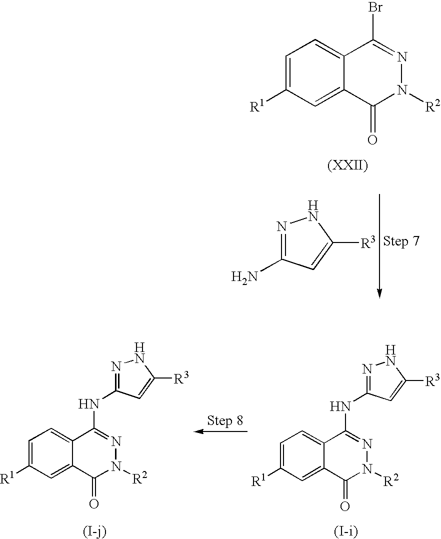

A preferred method for the synthesis of the compounds of formula I-i and 1-j starts from the Phthalazine dione of formula XV. Step 1 of the reaction sequence (scheme 6) is a two step process in which a dibromination is followed by a monohydrolysis, yielding the 4-bromo-alkylcarboxyphthalazinone derivatives of formula XVI. The first step (dibromination) is typically carried out without solvent, or in solvents like dichloromethane, dichloroethane, anisole, and mixtures thereof, at temperatures between 30° C. and 150° C. Typically used brominating reagents are phosphorus oxybromide, phosphorus pentabromide and phosphorus tribromide. The second step (monohydrolysis of the dibromide) is typically carried out in aqueous or anhydrous conditions in solvents such as water, aqueous lithium hydroxide, aqueous sodium hydroxide, aqueous potassium hydroxide, aqueous sodium hydrogen carbonate, aqueous sodium carbonate, aqueous potassium hydrogen carbonate, aqueous potassium carbonate, aqueous methanol, glacial acetic acid at temperatures between 20° C. and 110° C.

In step 2, scheme 6 the obtained compounds of formula XVI are converted into their corresponding esters of formula XVII, using methods well known to someone skilled in the art, e.g. esterification under acidic conditions. The reaction is typically carried out in protic solvents such as ethanol at temperatures between 20° C. and 100° C. Typically used acids are aqueous sulfuric acid, aqueous hydrochloric acid and aqueous acetic.

In step 3, scheme 6 the obtained compounds of formula XVII are converted into their corresponding tertiary amides of formula XVIII, using methods well known to someone skilled in the art, e.g. alkylation under basic conditions. The reaction is typically carried out in aprotic solvents such as tetrahydrofuran, N,N-dimethylformamide, dimethyl sulfoxide, N-methylpyrrolidinone and mixtures thereof at temperatures between −78° C. and 100° C. Typically used bases are sodium hydride, potassium hydride, sodium methoxide, potassium tert-butoxide, lithium hexamethyldisilazide, sodium hexamethyldisilazide, potassium hexamethyldisilazide.

In step 4, scheme 6 the obtained compounds of formula XVIII are converted into their corresponding alcohols of formula XIX, using methods well known to someone skilled in the art, e.g. reduction of esters to form alcohols. The reaction is typically carried out in aprotic solvents like tetrahydrofuran, dioxane, dichloromethane and mixtures thereof, or, in protic solvents such as methanol. The reaction is typically carried out at temperatures between 0° C. and 100° C. Typically used reducing reagents are lithium borohydride.

In step 5, scheme 6 the obtained compounds of formula XIX are converted into their corresponding alkyl bromides of formula XX, using methods well known to someone skilled in the art, e.g. functional group interconversion of alcohols into bromides. The reaction is typically carried out in solvents like acetonitrile, tetrahydrofuran, dioxane, dichloromethane and mixtures thereof, at temperatures between 0° C. and 100° C. Typically used brominating reagents are trimethylsilyl chloride or trimethylsilyl bromide in conjunction with lithium bromide.

In step 6, scheme 6 the obtained compounds of formula XX are converted into their corresponding thioethers of formula XXII, using methods well known to someone skilled in the art, e.g. alkylation of thiols. The reaction is typically carried out in solvents like N,N-dimethylformamide, tetrahydrofuran, N-methylpyrrolidinone, acetonitrile, acetone, dichloromethane and dichloroethane, at temperatures between 0° C. and 100° C. Typically used bases are potassium carbonate, sodium hydride, lithium hexamethyldisilazide, sodium hexamethyldisilazide and potassium hexamethyldisilazide in conjunction with nucleophiles such as thiols.

In step 7, scheme 6 the obtained compounds of formula XXII are converted into their corresponding amino pyrazoles of formula I-i, using methods well known to someone skilled in the art, e.g. palladium-mediated amination of imminobromides, vinylbromides or aryl bromides. The reaction is typically carried out in solvents such as tetrahydrofuran, toluene, alkanols such as methanol, ethanol, isopropanol, and mixtures thereof at temperatures between 40° C. and 110° C. Typically used bases are cesium carbonate, triethylamine, sodium tert-butoxide and appropriate ligated palladium (0) species can be generated using reagents such as palladium acetate, palladium dichloride, tris(dibenzylideneacetone)dipalladium, palladium tetrakis-triphenylphospine, bis-triphenylphosphinepalladium dichloride in conjunction with phosphine based ligands such as 2,2′-bi(phenylphosphino)-1,1′-binaphthyl, 4,5-Bis(diphenylphosphino)-9,9 dimethylxanthene and 2-(di-tert-butylphosphino)biphenyl.

In step 8, scheme 6 the obtained compounds of formula I-i are converted into their corresponding sulfones of formula I-j, using methods well known to someone skilled in the art, e.g. oxidation of thioethers to sulfones. The reaction is typically carried out in solvents such as tetrahydrofuran, toluene, alkanols such as methanol, ethanol, isopropanol and water and mixtures thereof at temperatures between 0° C. and 110° C. Typically used reagents are OXONE™ and meta-chloroperbenzoic acid.

### Scheme 7:

A preferred method for the synthesis of the derivatives of formula I, wherein R¹ is R⁵—X-alkylene-, X is —C(O)N(alkyl)- and R², R³ and R⁵ are defined as above in formula I, is described in scheme 7. The derivatives of formula I, wherein R¹ is wherein R¹ is R⁵—X-alkylene- and X is —C(O)N(alkyl)- are named I-k in scheme 7.

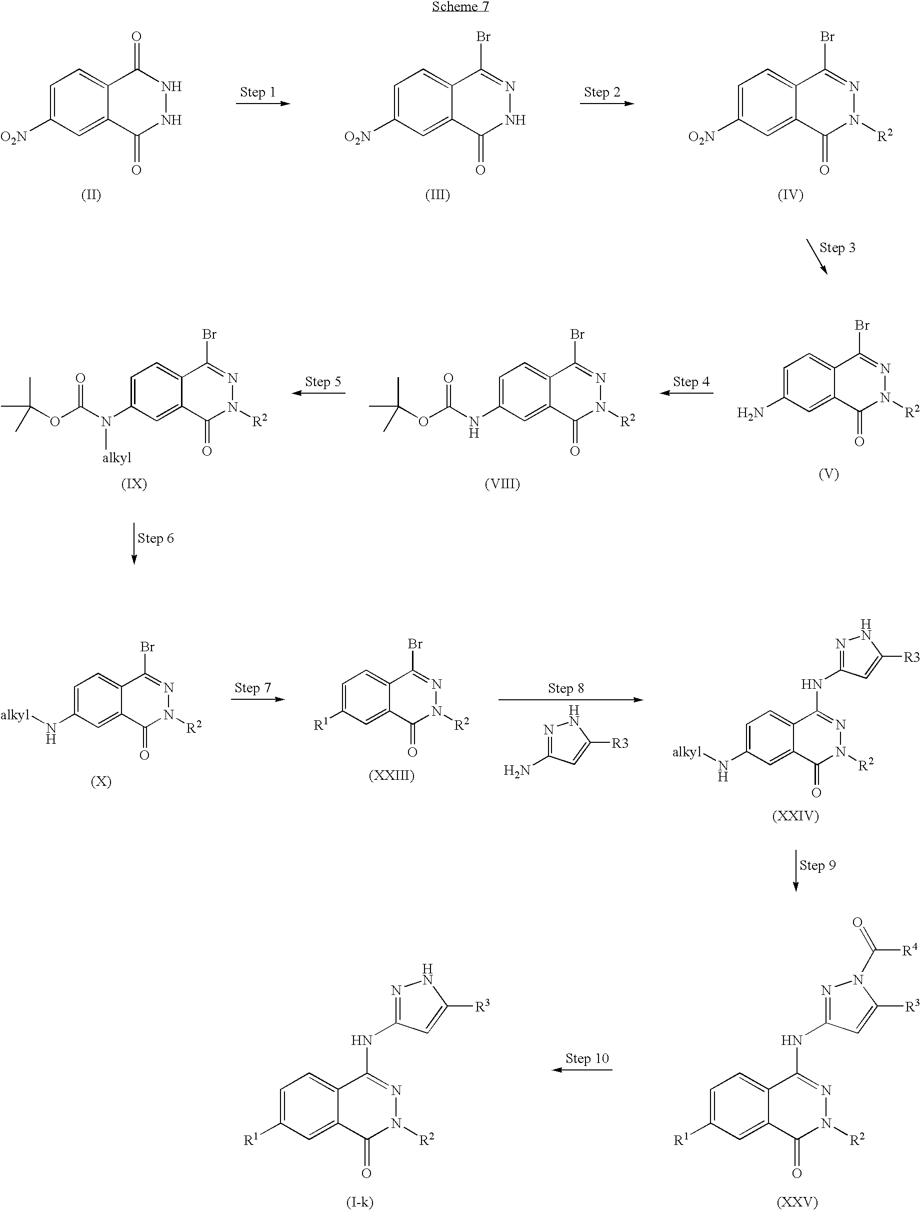

A preferred method for the synthesis of the compounds of formula I-k starts from the corresponding Phthalazine diones of formula II. Step 1 of the reaction sequence (scheme 7) is a two step process in which a dibromination is followed by a monohydrolysis, yielding the 4-bromo-nitrophthalazinone derivatives of formula III. The first step (dibromination) is typically carried out without solvent, or in solvents like dichloromethane, dichloroethane, anisole, and mixtures thereof, at temperatures between 30° C. and 150° C. Typically used brominating reagents are phosphorus oxybromide, phosphorus pentabromide and phosphorus tribromide. The second step (monohydrolysis of the dibromide) is typically carried out in aqueous or anhydrous conditions in solvents such as water, aqueous lithium hydroxide, aqueous sodium hydroxide, aqueous potassium hydroxide, aqueous sodium hydrogen carbonate, aqueous sodium carbonate, aqueous potassium hydrogen carbonate, aqueous potassium carbonate, aqueous methanol, glacial acetic acid at temperatures between 20° C. and 110° C.

In step 2, scheme 7 the obtained compounds of formula III are converted into their corresponding tertiary amides of formula IV, using methods well known to someone skilled in the art, e.g. alkylation under basic conditions. The reaction is typically carried out in aprotic solvents such as tetrahydrofuran, N,N-dimethylformamide, dimethyl sulfoxide, N-methylpyrrolidinone and mixtures thereof at temperatures between −78° C. and 100° C. Typically used bases are sodium hydride, potassium hydride, sodium methoxide, potassium tert-butoxide, lithium hexamethyldisilazide, sodium hexamethyldisilazide, potassium hexamethyldisilazide.

In step 3, scheme 7 the obtained compounds of formula IV are converted into their corresponding anilines of formula V, using methods well known to someone skilled in the art, e.g. aniline formation by the reduction of nitrobenzenes. The reaction is typically carried out in solvents like N,N-dimethylformamide, N-methylpyrrolidinone, acetonitrile, acetic acid, ethanol and methanol, and mixtures thereof, at temperatures between 20° C. and 100° C. Typically used reducing reagents are tin(II) chloride, tin(II) chloride monohydrate, iron trichloride.

In step 4, scheme 7 the obtained compounds of formula V are converted into their corresponding secondary carbamates of formula VIII, using methods well known to someone skilled in the art, e.g. tert-butyloxycarbonylation of amines. The reaction is typically carried out in solvents like N,N-dimethylformamide, N-methylpyrrolidinone, acetonitrile, dichloromethane and dichloroethane, at temperatures between 0° C. and 100° C. Typically used bases are imidazole, triethylamine, N,N-diisopropylethylamine, N,N-dimethylaminopyridine and sodium hydride in conjunction with reagents such as di-tert-butyl dicarbonate.

In step 5, scheme 7 the obtained compounds of formula VIII are converted into their corresponding tertiary carbamates of formula IX, using methods well known to someone skilled in the art, e.g. alkylation of secondary carbamates. The reaction is typically carried out in solvents like N,N-dimethylformamide, N-methylpyrrolidinone, acetonitrile, acetone, dichloromethane and dichloroethane, at temperatures between 0° C. and 100° C. Typically used bases are potassium carbonate, sodium hydride, lithium hexamethyldisilazide, sodium hexamethyldisilazide and potassium hexamethyldisilazide in conjunction with alkylating agents such as alkyl halides, alkyl mesylates and alkyl triflates.

In step 6, scheme 7 the obtained compounds of formula IX are converted into their corresponding secondary amines of formula X, using methods well known to someone skilled in the art, e.g. deprotection of acid labile protecting groups such as a tert-butyloxycarbonyl group. The reaction is typically carried out without solvent or in solvents like diethyl ether, dioxane, tetrahydrofuran, dichloromethane and dichloroethane or mixtures thereof, at temperatures between 0° C. and 40° C. Typically used acids are trifluoroacetic acid, trifluoromethane sulfonic acid, aqueous hydrochloric acid, aqueous sulfuric acid or anhydrous hydrogen chloride.

In step 7, scheme 7 the obtained compounds of formula X are converted into their corresponding amides of formula XXIII, using methods well known to someone skilled in the art, e.g. acylation of secondary amines. The reaction is typically carried out in solvents like N,N-dimethylformamide, N-methylpyrrolidinone, acetonitrile, acetone, dichloromethane and dichloroethane, at temperatures between 0° C. and 100° C. Typically used bases are potassium carbonate, sodium hydride, lithium hexamethyldisilazide, sodium hexamethyldisilazide and potassium hexamethyldisilazide in conjunction with acylating agents such as acid chlorides.

In step 8, scheme 7 the obtained compounds of formula XXIII are converted into their corresponding amino pyrazoles of formula XXIV, using methods well known to someone skilled in the art, e.g. palladium-mediated amination of imminobromides, vinylbromides or aryl bromides. The reaction is typically carried out in solvents such as tetrahydrofuran, toluene, alkanols such as methanol, ethanol, isopropanol, and mixtures thereof at temperatures between 40° C. and 110° C. Typically used bases are cesium carbonate, triethylamine, sodium tert-butoxide and appropriate ligated palladium (0) species can be generated using reagents such as palladium acetate, palladium dichloride, tris(dibenzylideneacetone)dipalladium, palladium tetrakis-triphenylphospine, bis-triphenylphosphinepalladium dichloride in conjunction with phosphine based ligands such as 2,2′-bi(phenylphosphino)-1,1′-binaphthyl, 4,5-Bis(diphenylphosphino)-9,9 dimethylxanthene and 2-(di-tert-butylphosphino)biphenyl.

In step 9, scheme 7 the obtained compounds of formula XXIV are converted into their corresponding pyrazoloamides of formula XXV, using methods well known to someone skilled in the art, e.g. acylation of secondary amines. The reaction is typically carried out in solvents like N,N-dimethylformamide, N-methylpyrrolidinone, acetonitrile, acetone, dichloromethane and dichloroethane, at temperatures between 0° C. and 100° C. Typically used bases are potassium carbonate, sodium hydride, lithium hexamethyldisilazide, sodium hexamethyldisilazide and potassium hexamethyldisilazide in conjunction with acylating agents such as acid chlorides.

In step 10, scheme 7 the obtained compounds of formula XXV are converted into their corresponding amides of formula I-k, using methods well known to someone skilled in the art, e.g. hydrolysis of pyrazoloamides. The reaction is typically carried out in protic solvents such as water, methanol and ethanol or aprotic solvents such as acetonitrile, dichloromethane, tetrahydrofuran, N,N-dimethylformamide, N-methylpyrrolidinone and mixtures thereof at temperatures between 0° C. and 80° C. Typically used bases are ammonia, potassium hydroxide, sodium hydroxide and lithium hydroxide.

Certain substituents on the groups R¹ may not be inert to the conditions of the synthesis sequences described above and may require protection by standard protecting groups known in the art. For instance, an amino or hydroxyl group may be protected as an acetyl or tert.-butoxycarbonyl derivative. Alternatively, some substituents may be derived from others at the end of the reaction sequence. For instance, a compound of formula I may be synthesized bearing a nitro-, an ethoxycarbonyl, a sulfonic acid substituent on the group R¹, which substituents are finally converted to an amino-, alkylamino-, dialkylamino-, alkylsulfonylamino, substituent, or to a carboxy substituent, by standard procedures.

Medicaments containing a compound of the present invention or a pharmaceutically acceptable salt thereof and a therapeutically inert carrier are an object of the present invention, as is a process for their production, which comprises bringing one or more compounds of the present invention and/or pharmaceutically acceptable salts and, if desired, one or more other therapeutically valuable substances into a galenical administration form together with one or more therapeutically inert carriers.

In accordance with the invention the compounds of the present invention as well as their pharmaceutically acceptable salts are useful in the control or prevention of illnesses. Based on their Aurora tyrosine kinase inhibition and/or their antiproliferative activity, said compounds are useful for the treatment of diseases such as cancer in humans or animals and for the production of corresponding medicaments. The dosage depends on various factors such as manner of administration, species, age and/or individual state of health.

An embodiment of the invention is a pharmaceutical composition, containing one or more compounds according to formula I, together with pharmaceutically acceptable excipients.

Another embodiment of the invention is a pharmaceutical composition containing one or more compounds of formula I as active ingredients together with pharmaceutically acceptable adjuvants for the treatment of diseases mediated by an inappropriate activation of Aurora family tyrosine kinases.

Another embodiment of the invention is a pharmaceutical composition, containing one or more compounds according to formula I as active ingredients together with pharmaceutically acceptable adjuvants for the inhibition of tumor growth.

Another embodiment of the invention is a pharmaceutical composition containing one or more compounds of formula I as active ingredients together with pharmaceutically acceptable adjuvants for the treatment of colorectal, breast, lung, prostate, pancreatic, gastric, bladder, ovarian, melanoma, neuroblastoma, cervical, kidney or renal cancers, leukemias or lymphomas.

Another embodiment of the invention is a pharmaceutical composition containing one or more compounds of formula I as active ingredients together with pharmaceutically acceptable adjuvants for the treatment of acute-myelogenous leukemia (AML, acute lymphocytic leukemia (ALL) and gastrointestinal stromal tumor (GIST).

Another embodiment of the invention is the use of one or more compounds of formula I for the manufacture of medicaments for the treatment of diseases mediated by an inappropriate activation of Aurora family tyrosine kinases.

Another embodiment of the invention is the use of a compound according to formula I, for the manufacture of corresponding medicaments for the inhibition of tumor growth.

Another embodiment of the invention is the use of a compound according to formula I, for the manufacture of corresponding medicaments for the treatment of colorectal, breast, lung, prostate, pancreatic, gastric, bladder, ovarian, melanoma, neuroblastoma, cervical, kidney or renal cancers, leukemias or lymphomas.

Another embodiment of the invention is the use of a compound according to formula I, for the manufacture of medicaments for the treatment of acute-myelogenous leukemia (AML, acute lymphocytic leukemia (ALL) and gastrointestinal stromal tumor (GIST).

Another embodiment of the invention is the use of the compounds of formula I as Aurora A tyrosine kinase inhibitors.

Another embodiment of the invention is the use of the compounds of formula I as anti-proliferating agents.

Another embodiment of the invention is the use of one or more compounds of formula I for the treatment of cancer.

The compounds according to the present invention may exist in the form of their pharmaceutically acceptable salts. The term “pharmaceutically acceptable salt” refers to conventional acid-addition salts that retain the biological effectiveness and properties of the compounds of formula I and are formed from suitable non-toxic organic or inorganic acids. Sample acid-addition salts include those derived from inorganic acids such as hydrochloric acid, hydrobromic acid, hydroiodic acid, sulfuric acid, sulfamic acid, phosphoric acid and nitric acid, and those derived from organic acids such as p-toluenesulfonic acid, naphthalenesulfonic acid, naphthalenedisulfonic acid, methanesulfonic acid, ethanesulfonic acid and the like. The chemical modification of a pharmaceutical compound (i.e. a drug) into a salt is a technique well known to pharmaceutical chemists to obtain improved physical and chemical stability, hygroscopicity, flowability and solubility of compounds. See, e.g. Stahl, P. H., and Wermuth, G., (editors), Handbook of Pharmaceutical Salts, Verlag Helvetica Chimica Acta (VHCA), Zürich, (2002), or Bastin, R. J., et al., Organic Proc. Res. Dev. 4 (2000) 427-435.

The compounds of formula I can contain one or several chiral centers and can then be present in a racemic or in an optically active form. The racemates can be separated according to known methods into the enantiomers. For instance, diastereomeric salts which can be separated by crystallization are formed from the racemic mixtures by reaction with an optically active acid such as e.g. D- or L-camphorsulfonic acid. Alternatively separation of the enantiomers can also be achieved by using chromatography on chiral HPLC-phases (HPLC: High Performance Liquid Chromatography) which are commercially available.

### Pharmacological Activity

The compounds of formula I and their pharmaceutically acceptable salts possess valuable pharmacological properties. It has been found that said compounds show activity as inhibitors of the Aurora kinase family and also show anti-proliferative activity. Consequently the compounds of the present invention are useful in the therapy and/or prevention of illnesses with known over-expression of kinases of the Aurora family, preferably Aurora A, especially in the therapy and/or prevention of illnesses mentioned above. The activity of the present compounds as inhibitors of the Aurora kinase family is demonstrated by the following biological assay:

### IC₅₀ Determination for Inhibitors of Aurora A

### Assay Principle

Aurora A is a serine threonine kinase involved in spindle assembly and chromosome segregation.

The assay is a typically ELISA-type assay where substrate (GST-Histone H3) is coupled to the assay-plate and is phosphorylated by the kinase. Phosphorylation is detected by a mouse anti-Phosphopeptid mAb and an HRP-labeled anti-mouse pAb. The assay is validated for IC₅₀-determination.

Kinase activities were measured by Enzyme-Linked Immunosorbent Assay (ELISA): Maxisorp 384-well plates (Nunc) were coated with recombinant fusion protein comprising residues 1-15 of Histone H3 fused to the N-terminus of Glutathione-S-Transferase. Plates were then blocked with a solution of 1 mg/mL I-block (Tropix cat# T2015—highly purified form of casein) in phosphate-buffered saline. Kinase reactions were carried out in the wells of the ELISA plate by combining an appropriate amount of mutant Aurora A kinase with test compound and 30 μM ATP. The reaction buffer was 10× Kinase Buffer (Cell Signaling cat #9802) supplemented with 1 μg/mL I-block. Reactions were stopped after 40 minutes by addition of 25 mM EDTA. After washing, substrate phosphorylation was detected by addition of anti-phospho-Histone H3 (Ser 10) 6G3 mAb (Cell Signaling cat #9706) and sheep anti-mouse pAb-HRP (Amersham cat# NA931V), followed by calorimetric development with TMB (3,3′,5,5′-tetramethylbenzidine from Kirkegaard & Perry Laboratories). After readout of the absorbance, IC₅₀ values were calculated using a non-linear curve fit (XLfit software (ID Business Solution Ltd., Guilford, Surrey, UK)). The results are shown in Table 1.

TABLE 1
Results

| | IC50 Aurora A kinase |
|---|---|
| Example No. | inhibition [μM] |
| A-4 | 0.010 |
| A-5 | 0.022 |
| C-2 | 0.010 |
| E-3 | 0.016 |
| D-1, E-1, E-2, F-1, F-2, G-1, G-2, G-3, H-1 | 0.001-0.100 |

### Antiproliferative Activity

The activity of the present compounds as antiproliferative agents is demonstrated by the following biological assay:

### CellTiter-Glo™ Assay in HCT 116 Cells

The CellTiter-Glo™ Luminescent Cell Viability Assay (Promega) is a homogeneous method of determining the number of viable cells in culture based on quantitation of the ATP present, which signals the presence of metabolically active cells.

HCT 116 cells (human colon carcinoma, ATCC-No. CC1-247) were cultivated in RPMI 1640 medium with GlutaMAX™ I (Invitrogen, Cat-No. 61870-010), 2.5% Fetal Calf Serum (FCS, Sigma Cat-No. F4135 (FBS)); 100 Units/ml penicillin/100 μg/ml streptomycin (=Pen/Strep from Invitrogen Cat. No. 15140). For the assay the cells were seeded in 384 well plates, 1000 cells per well, in the same medium. The next day the test compounds were added in various concentrations ranging from 30 μM to 0.0015 μM (10 concentrations, 1:3 diluted). After 5 days the CellTiter-Glo™ assay was done according to the instructions of the manufacturer (CellTiter-Glo™ Luminescent Cell Viability Assay, from Promega). In brief: the cell-plate was equilibrated to room temperature for approximately 30 minutes and than the CellTiter-Glo™ reagent was added. The contents were carefully mixed for 15 minutes to induce cell lysis. After 45 minutes the luminescent signal was measured in Victor 2, (scanning multiwell spectrophotometer, Wallac).

### Details:

1st. day:
Medium: RPMI 1640 with GlutaMAX™ I (Invitrogen, Cat-No. 61870), 5% FCS (Sigma Cat.-No. F4135), Pen/Strep (Invitrogen, Cat No. 15140).
HCT116 (ATCC-No. CC1-247): 1000 cells in 60 μl per well of 384 well plate (Greiner 781098, μClear-plate white)
After seeding incubate plates 24 h at 37° C., 5% CO₂
   2nd. day: Induction (Treatment with compounds, 10 concentrations):

In order to achieve a final concentration of 30 μM as highest concentration 3.5 μl of 10 mM compound stock solution were added directly to 163 μl media. Then step e) of the dilution procedure described below, was followed.

In order to achieve the second highest to the lowest concentrations, a serial dilution with dilution steps of 1:3 was followed according to the procedure (a-d) as described here below:
a) for the second highest concentration add 10 μl of 10 mM stock solution of compound to 20 μl dimethylsulfoxide (DMSO)
b) dilute 8× 1:3 (always 10 μl to 20 μl DMSO) in this DMSO dilution row (results in 9 wells with concentrations from 3333.3 μM to 0.51 μM)
c) dilute each concentration 1:47.6 (3.5 μl compound dilution to 163 μl media)
d) add 10 μl of every concentration to 60 μl media in the cell plate resulting in final concentration of DMSO: 0.3% in every well and resulting in 10 final concentration of compounds ranging from 30 μM to 0.0015 μM.
   Each compound is tested in triplicate.
   Incubate 120 h (5 days) at 37° C., 5% CO₂

### Analysis:

Add 30 μl CellTiter-Glo™ Reagent (prepared from CellTiter-Glo™ Buffer and CellTiter-Glo™ Substrate (lyophilized) purchased from Promega) per well,
shake 15 minutes at room temperature
incubate further 45 minutes at room temperature without shaking

### Measurement:

Victor 2 scanning multiwell spectrophotometer (Wallac), Luminescence mode (0.5 sec/read, 477 nm)
Determine IC50 using a non-linear curve fit (XLfit software (ID Business Solution Ltd., Guilford, Surrey, UK))

With all compounds a significant inhibition of HCT 116 cell viability was detected, which is exemplified by the compounds shown in Table 2.

TABLE 2
Results

| | IC50 HCT |
|---|---|
| Example No. | 116 [μM] |
| A-1 | 0.216 |
| A-4 | 0.791 |
| B-1 | 0.092 |
| C-1 | 0.296 |
| A-2, A-3, A-6, A-7, B-2, B-3, D-1, E-1, E-2, F-1, G-1, | 0.050-0.500 |
| A-5, A-6, C-2, E-3, F-2, G-2, G-3 | 0.500-2.500 |

The compounds according to this invention and their pharmaceutically acceptable salts can be used as medicaments, e.g. in the form of pharmaceutical compositions. The pharmaceutical compositions can be administered orally, e.g. in the form of tablets, coated tablets, dragées, hard and soft gelatine capsules, solutions, emulsions or suspensions. The administration can, however, also be effected rectally, e.g. in the form of suppositories, or parenterally, e.g. in the form of injection solutions.

The above-mentioned pharmaceutical compositions can be obtained by processing the compounds according to this invention with pharmaceutically inert, inorganic or organic carriers. Lactose, corn starch or derivatives thereof, talc, stearic acids or it's salts and the like can be used, for example, as such carriers for tablets, coated tablets, dragées and hard gelatine capsules. Suitable carriers for soft gelatine capsules are, for example, vegetable oils, waxes, fats, semi-solid and liquid polyols and the like. Depending on the nature of the active substance no carriers are, however, usually required in the case of soft gelatine capsules. Suitable carriers for the production of solutions and syrups are, for example, water, polyols, glycerol, vegetable oil and the like. Suitable carriers for suppositories are, for example, natural or hardened oils, waxes, fats, semi-liquid or liquid polyols and the like.

The pharmaceutical compositions can, moreover, contain preservatives, solubilizers, stabilizers, wetting agents, emulsifiers, sweeteners, colorants, flavorants, salts for varying the osmotic pressure, buffers, masking agents or antioxidants. They can also contain still other therapeutically valuable substances.

A pharmaceutical compositions comprise e.g. the following:

### a) Tablet Formulation (Wet Granulation):

| Item | Ingredients | Mg/tablet | | | |
|---|---|---|---|---|---|
| 1. | Compound of formula I | 5 | 25 | 100 | 500 |
| 2. | Lactose Anhydrous DTG | 125 | 105 | 30 | 150 |
| | (direct tabletting grade) | | | | |
| 3. | Sta-Rx 1500 (pre- | 6 | 6 | 6 | 30 |
| | gelatinized starch powder) | | | | |
| 4. | Microcrystalline Cellulose | 30 | 30 | 30 | 150 |
| 5. | Magnesium Stearate | 1 | 1 | 1 | 1 |
| | Total | 167 | 167 | 167 | 831 |

### Manufacturing Procedure:

1. Mix items 1, 2, 3 and 4 and granulate with purified water.
2. Dry the granules at 50° C.
3. Pass the granules through suitable milling equipment.
4. Add item 5 and mix for three minutes; compress on a suitable press.

### b) Capsule Formulation:

| Item | Ingredients | mg/capsule | | | |
|---|---|---|---|---|---|
| 1. | Compound of formula I | 5 | 25 | 100 | 500 |
| 2. | Hydrous Lactose | 159 | 123 | 148 | — |
| 3. | Corn Starch | 25 | 35 | 40 | 70 |
| 4. | Talc | 10 | 15 | 10 | 25 |
| 5. | Magnesium Stearate | 1 | 2 | 2 | 5 |
| | Total | 200 | 200 | 300 | 600 |

### Manufacturing Procedure:

1. Mix items 1, 2 and 3 in a suitable mixer for 30 minutes.
2. Add items 4 and 5 and mix for 3 minutes.
3. Fill into a suitable capsule.

### c) Micro Suspension

1. Weigh 4.0 g glass beads in custom made tube GL 25, 4 cm (the beads fill half of the tube).
2. Add 50 mg compound, disperse with spatulum and vortex.
3. Add 2 ml gelatin solution (weight beads:gelatin solution=2:1) and vortex.
4. Cap and wrap in aluminum foil for light protection.
5. Prepare a counter balance for the mill.
6. Mill for 4 hours, 20/s in a Retsch mill (for some substances up to 24 hours at 30/s).
7. Extract suspension from beads with two layers of filter (100 μm) on a filter holder, coupled to a recipient vial by centrifugation at 400 g for 2 min.
8. Move extract to measuring cylinder.
9. Repeat washing with small volumes (here 1 ml steps) until final volume is reached or extract is clear.
10. Fill up to final volume with gelatin and homogenize.

The following examples are provided to aid the understanding of the present invention, the true scope of which is set forth in the appended claims. It is understood that modifications can be made in the procedures set forth without departing from the spirit of the invention.

### EXAMPLES

### Method A

### Example A-1

### 2-Isopropyl-4-(5-methyl-1H-pyrazol-3-ylamino)-7-(2-methylsulfanyl-ethoxy)-2H-phthalazin-1-one

### 7-Nitro-2,3-dihydro-phthalazine-1,4-dione

Hydrazine hydrate (26.6 g, 0.53 mol) was added in one portion to a stirred mixture of 4-nitrophthalic anhydride (100 g, 0.52 mol), in acetic acid (1.0 L) at room temperature. The mixture was heated to 120° C. for 2 hours and then allowed to cool to room temperature. The solid was filtered, washed with water (250 ml) and dried under vacuum at 50° C. for 20 hours to give the nitrophthalazinone (95 g, 88% yield). Tr=0.85 min m/z (ES⁺) (M+H⁺) 208

### 6-Nitro-4-bromo-2H-phthalazin-1-one and 7-Nitro-4-bromo-2H-phthalazin-1-one

7-Nitro-2,3-dihydro-phthalazine-1,4-dione (95.0 g, 0.46 mol) was suspended in dichloroethane (1.0 L) and phosphorus pentabromide (789.0 g, 1.83 mol) was added in three portions and the reaction heated to reflux for 24 hours. The reaction was cooled to room temperature and poured onto ice (2.5 kg) and the resulting precipitate filtered and washed with water to give the crude product (160.0 g). This crude material was suspended in acetic acid (1.60 L) and heated to 125° C. for 2 hours. The reaction was cooled to room temperature and poured onto ice (1.5 kg) and the resulting precipitate filtered. The solid was washed with water and dried to give the title compounds (84 g, 68% yield, 1:1 mixture of isomers) as a yellow solid. 7-Nitro: δ_{H} (400 MHz, DMSO), 13.29 (1H), 8.83 (1H, d), 8.79 (1H, dd), 8.61 (1H, dd), 8.54 (1H, d), 8.46 (1H, d), 8.16 (d) Tr=1.11 min, m/z (ES⁺) (M+H)⁺ 269 & 271

### 7-Nitro-2-isopropyl-4-bromo-2H-phthalazin-1-one

A mixture of 6-Nitro-4-bromo-2H-phthalazin-1-one and 7-Nitro-4-bromo-2H-phthalazin-1-one (84 g, 0.31 mol) was dissolved in DMF (400 ml). To this was added NaH (60%, 7.5 g, 0.31 mol) as a DMF suspension (200 ml). The mixture was stirred at room temperature for 30 minutes then 2-bromo-propane (7.7 g, 62 mmol) was added in one portion as a solution in DMF (250 ml). The reaction mixture was stirred for 24 hours whereupon LC-MS showed 40% starting material remaining. To this was added NaH (3.75 g, 0.15 mol) and the reaction stirred for a further 24 hours. The DMF was removed under vacuum and the resulting crude material purified by successive column chromatography (elution: 92% heptane, 8% ethyl acetate) to give the title compound (38.8 g, 40% yield) as a light yellow solid.

δ_{H} (400 MHz, DMSO), 8.88 (1H, d), 8.87 (1H, dd), 8.16 (1H, d), 5.19 (1H, m), 1.13 (6H, d).

### 7-Amino-2-isopropyl-4-bromo-2H-phthalazin-1-one

7-Nitro-2-isopropyl-4-bromo-2H-phthalazin-1-one (4.6 g, 0.015 mol) was dissolved in a 5:1 mixture of ethanol and water (150 ml). To this solution was added iron powder (2.14 g, 0.039 mol) and concentrated hydrochloric acid (1 ml), the mixture was heated to 80° C. for 3 hours. After this time, the reaction mixture was cooled to room temperature and filtered through a pad of celite, the celite was washed with ethanol (100 ml), and the solution was concentrated under vacuum to give the title compound (4.2 g, 98% yield) as a white solid.

δ_{H} (400 MHz, DMSO), 7.56 (1H, d), 7.28 (1H, s), 7.13 (1H, d), 6.47 (2H, s), 5.24-5.09 (1H, m), 1.23 (6H, d) Tr=1.34 min m/z (ES⁺) (M+H)⁺ 282, 284

### 7-Hydroxy-2-Isopropyl-4-bromo-2H-phthalazin-1-one

Concentrated sulfuric acid (17 ml) was added slowly to a solution of 7-amino-2-Isopropyl-4-bromo-2H-phthalazin-1-one (4.6 g, 0.016 mol) in acetic acid (50 ml). The reaction mixture was cooled to 0° C. and a solution of sodium nitrite (1.52 g, 0.022 mol) in water (10 ml) was added dropwise. The reaction mixture was stirred for a further 20 minutes at 0° C. prior to the addition of urea (0.55 g, 0.009 mol) and cold water (50 ml). The reaction mixture was then added carefully to a refluxing mixture of sulfuric acid (28 ml) in water (115 ml) and the reaction was stirred for a further 10 minutes at reflux before being allowed to cool to room temperature. Upon standing, an orange precipitate was observed, which was collected by filtration and washed with water to give the title compound (4.22 g, 93% yield) as an orange powder.

δ_{H} (400 MHz, DMSO), 11.18 (1H, br s), 7.93 (1H, d), 7.71 (1H, d), 7.53 (1H, dd), 5.34-5.26 (1H, m), 1.42 (6H, d)

### 4-Bromo-2-isopropyl-7-(2-methylsulfanyl-ethoxy)-2H-phthalazin-1-one

To a solution of 7-hydroxy-2-isopropyl-4-bromo-2H-phthalazin-1-one (0.8 g, 0.0028 mol) in DMF (8 ml), was added potassium carbonate (2 g, 0.014 mol). After 5 minutes, 2-chloroethyl methyl sulfide (0.34 g, 0.0028 mol) was added and the solution was heated to 100° C. for 24 hours. After this time LC-MS indicated the complete consumption of starting material and the mixture was cooled, concentrated under vacuum and purified by flash column chromatography (elution: 70% heptane, 30% ethyl acetate) to give the title compound (0.3 g, 24% yield) as a white solid.

### 2-Isopropyl-4-(5-methyl-1H-pyrazol-3-ylamino)-7-(2-methylsulfanyl-ethoxy)-2H-phthalazin-1-one (A-1)

Degassed toluene (6 ml) and ethanol (3 ml) were added in one portion to a mixture of 4-bromo-2-isopropyl-7-(2-methylsulfanyl-ethoxy)-2H-phthalazin-1-one (0.3 g, 0.8 mmol), sodium t-butoxide (0.112 g, 1.2 mmol), 3-amino-5-methylpyrazole (0.107 g, 1.2 mmol), tris-(dibenzylideneacetone)-dipalladium (0.038 g, 0.042 mmol) and 2-(di-t-butylphosphino)-biphenyl (0.025 g, 0.084 mmol) under nitrogen. The reaction mixture was heated to 100° C. for 20 hours with stirring and then cooled to room temperature. Diethyl ether (10 ml) was added and the precipitated solid was filtered to give the crude product as a grey solid. Flash column chromatography (elution: 95% ethyl acetate, 5% methanol) afforded the title compound as a white solid (0.070 g, 7% yield).

### Example A-1

### 2-Isopropyl-4-(5-methyl-1H-pyrazol-3-ylamino)-7-(2-methylsulfanyl-ethoxy)-2H-phthalazin-1-one

δ_{H} (400 MHz, DMSO) 11.95-11.83 (1H, m), 9.10 (1H, s), 8.39 (1H, d), 7.68 (1H, d), 7.46 (1H, dd), 6.33 (1H, s), 5.29-5.20 (1H, m), 4.33 (2H, t), 2.91 (2H, t), 2.24 (3H, s), 2.18 (3H, s), 1.32 (6H, d) Tr=1.77 min, m/z (ES⁺) (M+H)⁺ 374.26.

### Example A-2

### 2-Isopropyl-7-(2-methoxy-ethoxy)-4-(5-methyl-1H-pyrazol-3-ylamino)-2H-phthalazin-1-one

δ_{H} (400 MHz, DMSO) 9.18 (1H, s), 8.36 (1H, d), 7.68 (1H, d), 7.47 (1H, dd), 6.34 (1H, s), 5.30-5.18 (1H, m), 4.28 (2H, t), 3.72 (2H, t), 3.33 (3H, s), 2.25 (3H, s), 1.32 (6H, d) Tr=1.12 min, m/z (ES⁺) (M+H)⁺ 358.39.

### Example A-3

### 3-[3-Isopropyl-1-(5-methyl-1H-pyrazol-3-ylamino)-4-oxo-3,4-dihydro-phthalazin-6-yloxy]-propane-1-sulfonic acid dimethylamide

δ_{H} (400 MHz, DMSO) 11.89 (1H, br s), 9.10 (1H, s), 8.39 (1H, d), 7.68 (1H, d), 7.47 (1H, dd), 6.33 (1H, s), 5.29-5.19 (1H, m), 4.27 (2H, t), 3.27-3.21 (2H, m), 2.80 (6H, s), 2.24 (3H, s), 2.21-2.14 (2H, m), 1.31 (6H, d) Tr=1.23 min, m/z (ES⁺) (M+H)⁺ 449.19.

### Example A-4

### 2-Isopropyl-4-(5-methyl-1H-pyrazol-3-ylamino)-7-[3-(morpholine-4-sulfonyl)-propoxy]-2H-phthalazin-1-one

δ_{H} (400 MHz, DMSO) 11.88 (1H, br s), 9.10 (1H, s), 8.39 (1H, d), 7.68 (1H, d), 7.47 (1H, dd), 6.33 (1H, s), 5.30-5.18 (1H, m), 4.27 (2H, t), 3.66-3.62 (4H, m), 3.31-3.25 (2H, m), 2.24 (3H, s), 2.22-2.15 (2H, m), 1.32 (6H, d) Tr=1.60 min, m/z (ES⁺) (M+H)⁺ 491.22.

### Example A-5

### 7-(2-Dimethylamino-ethoxy)-2-isopropyl-4-(5-methyl-1H-pyrazol-3-ylamino)-2H-phthalazin-1-one

δ_{H} (400 MHz, DMSO) 8.41 (1H, d), 7.67 (1H, d), 7.45 (1H, dd), 6.32 (1H, s), 5.32-5.16 (1H, m), 4.23 (2H, t), 2.68 (2H, t), 2.23 (9H, s), 1.32 (6H, d) Tr=1.38 min, m/z (ES⁺) (M+H)⁺ 371.28.

### Example A-6

### 2-Isopropyl-7-(2-methanesulfinyl-ethoxy)-4-(5-methyl-1H-pyrazol-3-ylamino)-2H-phthalazin-1-one

To a solution of 2-isopropyl-4-(5-methyl-1H-pyrazol-3-ylamino)-7-(2-methylsulfanyl-ethoxy)-2H-phthalazin-1-one (0.077 g, 0.23 mmol) in dichloromethane (2 ml), was added m-chloroperoxybenzoic acid (0.040 g, 0.23 mmol) portion wise. The solution was stirred at room temperature for 1 hour. After this time LC-MS indicated complete consumption of starting material and the solvent was removed under vacuum. Preparative thin layer chromatography (elution: 50% ethyl acetate, 50% ethanol) afforded the title compound (0.015 g, 14% yield) as a pale yellow solid.

δ_{H} (400 MHz, DMSO) 11.93 (1H, br s), 9.16 (1H, br s), 8.42 (1H, d), 7.72 (1H, d), 7.50 (1H, dd), 6.33 (1H, s), 5.29-5.20 (1H, m), 4.62-4.55 (1H, m), 4.52-4.43 (1H, m), 3.17-3.09 (2H, m), 2.67 (3H, s), 2.23 (3H, s), 1.32 (6H, d) Tr=1.02 min, m/z (ES⁺) (M+H)⁺ 390.26.

### Example A-7

### 2-Isopropyl-7-(2-methanesulfonyl-ethoxy)-4-(5-methyl-1H-pyrazol-3-ylamino)-2H-phthalazin-1-one

To a solution of 2-isopropyl-4-(5-methyl-1H-pyrazol-3-ylamino)-7-(2-methylsulfanyl-ethoxy)-2H-phthalazin-1-one (0.077 g, 0.23 mmol) in dichloromethane (2 ml), was added m-chloroperoxybenzoic acid (0.080 g, 0.46 mmol) portion wise. The solution was stirred at room temperature overnight. After this time LC-MS indicated complete consumption of starting material and the solvent was removed in vacuo. Preparative thin layer chromatography (elution: 50% ethyl acetate, 50% ethanol) afforded the title compound (0.010 g, 9% yield) as a white solid.

δ_{H} (400 MHz, DMSO) 11.90 (1H, s), 9.12 (1H, s), 8.42 (1H, d), 7.72 (1H, d), 7.53 (1H, dd), 6.35 (1H, s), 5.31-5.16 (1H, m), 4.54 (2H, t), 3.70 (2H, t), 3.11 (3H, s), 2.24 (3H, s), 1.32 (6H, d) Tr=1.59 min, m/z (ES⁺) (M+H)⁺ 406.22.

### Method B

### Example B-1

### 2-Isopropyl-7-[methyl-(2-methylsulfanyl-ethyl)-amino]-4-(5-methyl-1H-pyrazol-3-ylamino)-2H-phtalazin-1-one

### (1-Bromo-3-isopropyl-4-oxo-3,4-dihydro-phthalazin-6-yl)-carbamic acid tert-butyl ester

7-Amino-2-isopropyl-4-bromo-2H-phthalazin-1-one (1.88 g, 6.7 mmol) was dissolved in DMF (20 ml). To this was added NaH (60%, 0.8 g, 20.1 mmol) as a suspension in DMF (5 ml). The mixture was stirred at room temperature for 30 minutes then Boc₂O (4.36 g, 20.1 mmol) was added in one portion as a solution in DMF (5 ml) and the reaction mixture was heated to 70° C. for 3 hours. After this time, the reaction mixture was cooled to room temperature and water (20 ml) was added cautiously, the mixture was extracted with ethyl acetate (3×50 ml), the organic layers were combined, dried (MgSO₄), filtered and concentrated under vacuum.

The residue was dissolved in a 1:1 mixture of THF/ethanol (10 ml) and aqueous NaOH (50% by weight solution, 10 ml) was added in one portion, the reaction mixture was stirred vigorously for 30 minutes. After this time, the mixture was partitioned between water (20 ml) and ethyl acetate (50 ml). The organic layer was dried (MgSO₄), filtered and concentrated to give the title compound (2.2 g, 88% yield) as a light brown solid.

δ_{H} (400 MHz, DMSO), 8.32 (1H, d), 8.19 (1H, s), 7.88 (1H, d), 7.41 (1H, s), 5.46-5.31 (1H, m), 1.52 (9H, s), 1.41 (6H, d) Tr=1.73 min, m/z (ES⁺) (M+H)⁺ 382.22.

### 4-Bromo-2-isopropyl-7-methylamino-2H-phthalazin-1-one

(1-Bromo-3-isopropyl-4-oxo-3,4-dihydro-phthalazin-6-yl)-carbamic acid tert-butyl ester (2.2 g, 5.7 mmol) was dissolved in THF (10 ml). To this was added NaH (60%, 0.34 g, 8.6 mmol) as a suspension in THF (5 ml). The mixture was stirred at room temperature for 30 minutes then methyl iodide (1.4 ml, 23.0 mmol) was added in one portion as a solution in THF (5 ml) and the reaction mixture was stirred at room temperature for 3 hours. After this time, the reaction mixture was cooled to room temperature and water (20 ml) was added cautiously, the mixture was extracted with ethyl acetate (3×50 ml), the organic layers were combined, dried (MgSO₄), filtered and concentrated under vacuum.

The residue was dissolved in a 20% TFA/DCM solution (10 ml) and the reaction mixture was stirred at room temperature for 2 hours. After this time, the reaction mixture was concentrated under vacuum to afford a brown oil. Heptane (20 ml) was added, and the mixture was concentrated under vacuum. Ether (10 ml) was added to the residue and the resulting precipitate was filtered and dried under vacuum to afford the title compound (1.14 g, 68% yield) as a light brown solid. Tr=1.50 min, m/z (ES⁺) (M+H)⁺ 296.16.

### 4-Bromo-2-isopropyl-7-[methyl-(2-methylsulfanyl-ethyl)-amino]-2H-phthalazin-1-one

4-Bromo-2-isopropyl-7-methylamino-2H-phthalazin-1-one (0.095 g, 0.32 mmol) was dissolved in DMF (5 ml). To this was added NaH (60%, 0.015 g, 0.38 mmol) as a suspension in DMF (2 ml). The mixture was stirred at room temperature for 30 minutes then chloroethyl methyl sulfide (0.042 g, 0.38 mmol) was added in one portion as a solution in DMF (1 ml) and the reaction mixture was heated to 70° C. for 24 hours. After this time, the reaction mixture was cooled to room temperature and water (10 ml) was added cautiously, the mixture was extracted with ethyl acetate (3×10 ml), the organic layers were combined, dried (MgSO₄), filtered and concentrated under vacuum. Flash column chromatography (elution: 70% hexane, 30% ethyl acetate) gave the title compound (0.021 g, 18% yield) as a white solid.

δ_{H} (400 MHz, CDCl₃), 7.73 (1H, d), 7.48 (1H, d), 7.13 (1H, d), 5.41-5.29 (1H, m), 3.71 (2H, t), 3.18 (3H, s), 3.15 (3H, s), 2.73 (2H, t), 1.41 (6H, d).

This material was then used in the Buchwald reaction as described in Method A to give the corresponding 2-Isopropyl-7-[methyl-(2-methylsulfanyl-ethyl)-amino]-4-(5-methyl-1H-pyrazol-3-ylamino)-2H-phtalazin-1-one (B-1).

### Example B-1

### 2-Isopropyl-7-[methyl-(2-methylsulfanyl-ethyl)-amino]-4-(5-methyl-1H-pyrazol-3-ylamino)-2H-phtalazin-1-one

δ_{H} (400 MHz, DMSO), 9.11 (1H, s), 8.19 (1H, d), 7.35 (1H, d), 7.28 (1H, dd), 6.34 (1H, s), 5.29-5.19 (1H, m), 3.71 (2H, t), 3.09 (3H, s), 2.69 (2H, t), 2.25 (3H, s), 2.14 (3H, s), 1.31 (6H, d) Tr=1.80 min, m/z (ES⁺) (M+H)⁺ 387.26.

### Example B-2

### 2-Isopropyl-7-[(2-methoxy-ethyl)-methyl-amino]-4-(5-methyl-1H-pyrazol-3-ylamino)-2H-phthalazin-1-one

δ_{H} (400 MHz, DMSO), 11.83 (1H, s), 8.87 (1H, s), 8.21 (1H, d), 7.34 (1H, d), 7.25 (1H, d), 6.34 (1H, s), 5.30-5.18 (1H, m), 3.66 (2H, t), 3.53 (2H, t), 3.25 (3H, s), 3.06 (3H, s), 2.23 (3H, s), 1.30 (6H, d) Tr=1.69 min, m/z (ES⁺) (M+H)⁺ 371.32.

### Example B-3

### 7-[(2-Dimethylamino-ethyl)-methyl-amino]-2-isopropyl-4-(5-methyl-1H-pyrazol-3-ylamino)-2H-phthalazin-1-one

δ_{H} (400 MHz, DMSO), 11.83 (1H, br s), 8.90 (1H, br s), 8.21 (1H, d), 7.33 (1H, d), 7.22 (1H, d), 6.33 (1H, br s), 5.29-5.17 (1H, m), 3.61-3.52 (2H, m), 3.05 (3H, t), 2.41 (2H, t), 2.23 (3H, s), 2.20 (6H, s), 1.30 (6H, d) Tr=1.01 min, m/z (ES⁺) (M+H)⁺ 384.22.

### Method C

### Example C-1

### 2-Isopropyl-4-(5-methyl-1H-pyrazol-3-ylamino)-7-(2-methylsulfanyl-ethylsulfanyl)-2H-phthalazin-1-one

### 7-Mercapto-2-isopropyl-4-bromo-2H-phthalazin-1-one

Concentrated sulfuric acid (5 ml) was added dropwise to a solution of 7-amino-2-isopropyl-4-bromo-2H-phthalazin-1-one (1.5 g, 5.3 mmol) in acetic acid (15 ml) and the solution was cooled to 0° C. A solution of sodium nitrite (0.5 g, 7.4 mmol) in water (2.5 ml) was added dropwise and the reaction mixture was stirred at 0° C. for 20 minutes, after which time urea (0.17 g, 2.8 mmol) was added in one portion. The reaction mixture was then added dropwise to a solution of potassium ethyl xanthate (6 g, 37.7 mmol) in water (7.5 ml) and the mixture was heated to 80° C. for 30 minutes. After this time, the reaction mixture was cooled to room temperature and DCM (100 ml) was added. The organic layer was separated, dried (MgSO₄), filtered and concentrated under vacuum.

The residue was taken up in THF (10 ml), NaOH (4.95 g, 0.12 mmol) was added in one portion and the mixture was heated to reflux for 24 hours. The mixture was then cooled to room temperature and the suspension was acidified to pH 2 with concentrated HCl. DCM (100 ml) was added, the organic layer was separated and was subsequently washed with HCl (1M, 20 ml) and water (20 ml). The organic layer was extracted with NaOH (1M, 200 ml), the aqueous layer was separated and acidified to pH 1 with concentrated HCl. The mixture was extracted with DCM (2×50 ml), the organic layers were combined, dried (MgSO₄), filtered and concentrated under vacuum to give the title compound (0.77 g, 48% yield) as a light brown solid which was taken on directly without further purification.

### 4-Bromo-2-isopropyl-7-(2-methylsulfanyl-ethylsulfanyl)-2H-phthalazin-1-one

To a solution of crude 7-mercapto-2-isopropyl-4-bromo-2H-phthalazin-1-one (0.40 g, 1.3 mmol) in DMF (8 ml), was added NaH (60%, 0.064 g, 1.6 mmol) portion-wise. After stirring for 5 minutes, chloroethyl methylsulfide (0.17 g, 1.6 mmol) was added dropwise. The mixture was heated to 60° C. for two hours, after which time the mixture was concentrated under vacuum and the residue was subjected to flash column chromatography (elution: 90% heptane, 10% ethyl acetate) to give the title compound (0.44 g, 54% yield) as a white solid.

δ_{H} (400 MHz, DMSO), 8.03 (1H, d), 7.92 (1H, d), 7.81 (1H, d), 5.26-5.15 (1H, m), 3.41 (2H, t), 2.78 (2H, t), 2.15 (3H, s), 1.36 (6H, d).

This material was then used in the Buchwald reaction as described in Method A to give the corresponding 2-isopropyl-4-(5-methyl-1H-pyrazol-3-ylamino)-7-(2-methylsulfanyl-ethylsulfanyl)-2H-phthalazin-1-one (C-1).

### Example C-1

### 2-Isopropyl-4-(5-methyl-1H-pyrazol-3-ylamino)-7-(2-methylsulfanyl-ethylsulfanyl)-2H-phthalazin-1-one

Tr=1.33 min, m/z (ES⁺) (M+H)⁺ 390.23.

### Example C-2

### 2-Isopropyl-7-(2-methanesulfonyl-ethanesulfonyl)-4-(5-methyl-1H-pyrazol-3-ylamino)-2H-phthalazin-1-one

Oxone (0.12 g, 0.07 mmol) was added in one portion to a stirred solution of 2-isopropyl-4-(5-methyl-1H-pyrazol-3-ylamino)-7-(2-methylsulfanyl-ethylsulfanyl)-2H-phthalazin-1-one (0.013 g, 0.03 mmol) in a 4:1 mixture of dioxane/water (1.2 ml) and the reaction mixture was stirred at room temperature for 1 hour. The reaction mixture was diluted with water (5 ml) and the solution was extracted with ethyl acetate (3×75 ml), the organic layers were combined, dried (MgSO₄), filtered and concentrated under vacuum to give the title compound (0.008 g, 57% yield) as a white solid.

δ_{H} (400 MHz, DMSO) 9.69 (1H, br s), 8.75 (1H, d), 8.71 (1H, d), 8.47 (1H, dd), 6.40 (1H, s), 5.31-5.22 (1H, m), 3.95-3.89 (2H, m), 3.48-3.42 (2H, m), 3.06 (3H, s), 2.29 (3H, s), 1.36 (6H, d) Tr=1.61 min, m/z (ES⁺) (M+H)⁺ 454.10.

### Method D

### Example D-1

### 7-(2-Dimethylamino-ethoxy)-2-isopropyl-4-(5-methyl-1H-pyrazol-3-ylamino)-2H-phthalazin-1-one

### (1-Bromo-3-isopropyl-4-oxo-3,4-dihydro-phthalazin-6-yl)-carbamic acid tert-butyl ester

7-Amino-2-isopropyl-4-bromo-2H-phthalazin-1-one (1.88 g, 6.7 mmol) was dissolved in DMF (20 ml). To this was added NaH (60%, 0.8 g, 20.1 mmol) as a suspension in DMF (5 ml). The mixture was stirred at room temperature for 30 minutes then Boc₂O (4.36 g, 20.1 mmol) was added in one portion as a solution in DMF (5 ml) and the reaction mixture was heated to 70° C. for 3 hours. After this time, the reaction mixture was cooled to room temperature and water (20 ml) was added cautiously, the mixture was extracted with ethyl acetate (3×50 ml), the organic layers were combined, dried (MgSO₄), filtered and concentrated under vacuum.

The residue was dissolved in a 1:1 mixture of THF/ethanol (10 ml) and aqueous NaOH (50% by weight solution, 10 ml) was added in one portion, the reaction mixture was stirred vigorously for 30 minutes. After this time, the mixture was partitioned between water (20 ml) and ethyl acetate (50 ml). The organic layer was dried (MgSO₄), filtered and concentrated to give the title compound (2.2 g, 88% yield) as a light brown solid.

δ_{H} (400 MHz, DMSO), 8.32 (1H, d), 8.19 (1H, s), 7.88 (1H, d), 7.41 (1H, s), 5.46-5.31 (1H, m), 1.52 (9H, s), 1.41 (6H, d) Tr=1.73 min, m/z (ES⁺) (M+H)⁺ 382.22.

### 7-(2-Dimethylamino-ethoxy)-2-isopropyl-4-(5-methyl-1H-pyrazol-3-ylamino)-2H-phthalazin-1-one

(1-Bromo-3-isopropyl-4-oxo-3,4-dihydro-phthalazin-6-yl)-carbamic acid tert-butyl ester (0.75 g, 1.96 mmol) was dissolved in DMF (10 ml). To this was added NaH (60%, 0.2 g, 4.9 mmol) as a suspension in DMF (5 ml). The mixture was stirred at room temperature for 30 minutes then 1-bromo-2-methoxyethane (0.4 g, 2.9 mmol) was added in one portion as a solution in DMF (5 ml) and the reaction mixture was stirred at room temperature for 3 hours. After this time, the reaction mixture was cooled to room temperature and water (20 ml) was added cautiously, the mixture was extracted with ethyl acetate (3×50 ml), the organic layers were combined, dried (MgSO₄), filtered, concentrated under vacuum and the residue subjected to flash column chromatography (elution: 60% heptane, 40% ethyl acetate) to afford (1-bromo-3-isopropyl-4-oxo-3,4-dihydro-phthalazin-6-yl)-(2-dimethylamino-ethyl)-carbamic acid tert-butyl ester (0.2 g, 23% yield) as a white solid.

This material was then used in the Buchwald reaction as described in Method A to give the corresponding 2-isopropyl-7-[methyl-(2-methylsulfanyl-ethyl)-amino]-4-(5-methyl-1H-pyrazol-3-ylamino)-2H-phtalazin-1-one.

The residue was dissolved in a 20% TFA/DCM solution (5 ml) and the reaction mixture was stirred at room temperature for 2 hours. After this time, the reaction mixture was concentrated under vacuum to afford a brown oil. Heptane (2 ml) was added, and the mixture was concentrated under vacuum. Ether (1 ml) was added to the residue and the resulting precipitate was filtered and dried under vacuum to afford the title compound (0.061 g, 6% yield) as a pale yellow solid.

### Example D-1

### 7-(2-Dimethylamino-ethoxy)-2-isopropyl-4-(5-methyl-1H-pyrazol-3-ylamino)-2H-phthalazin-1-one

δ_{H} (400 MHz, DMSO), 9.41 (1H, br s), 8.95 (1H, s), 8.18 (1H, d), 7.35 (1H, d), 7.14 (1H, dd), 6.33 (1H, s), 5.29-5.17 (1H, m), 3.60-3.53 (2H, m), 3.33-3.26 (2H, m), 2.88-2.84 (6H, m), 2.24 (3H, s), 1.31 (6H, d) Tr=1.50 min, m/z (ES⁺) (M+H)⁺ 370.38.

### Method E

### Example E-1

### 7-Cyclopropylmethoxymethyl-2-isopropyl-4-(5-methyl-1H-pyrazol-3-ylamino)-2H-phthalazin-1-one

### 1,4-Dioxo-1,2,3,4-tetrahydro-phthalazine-6-carboxylic acid

Hydrazine hydrate (26 g, 0.52 mol) was added in one portion to a stirred mixture of 1,2,4-benzenetricarboxylic anhydride (100 g, 0.52 mol), in acetic acid (1.0 L) at room temperature. The mixture was heated to 120° C. for 2 hours and then allowed to cool to room temperature. The solid was filtered, washed with water (250 ml) and dried in the under vacuum at 50° C. for 20 hours to give the title compound (91 g, 85% yield).

### 1-Bromo-4-oxo-3,4-dihydro-phthalazine-6-carboxylic acid

1,4-Dioxo-1,2,3,4-tetrahydro-phthalazine-6-carboxylic acid (91.0 g, 0.44 mol) was suspended in dichloroethane (1.0 L) and phosphorus pentabromide (761.0 g, 1.77 mol) was added in three portions and the reaction heated to reflux for 24 hours. The reaction was cooled to room temperature and poured onto ice (2.5 kg) and the resulting precipitate filtered and washed with water to give the crude product (130.0 g).

This crude material was suspended in acetic acid (1.6 L) and heated to 125° C. for 2 hours. The reaction was cooled to room temperature and poured onto ice (1.5 kg) and the resulting precipitate filtered. The solid was washed with water and dried to give the title compound (85 g, 73% yield) as a yellow solid. Tr=0.94 min, m/z (ES⁺) (M+H)⁺ 310 & 312

### 1-Bromo-4-oxo-3,4-dihydro-phthalazine-6-carboxylic acid ethyl ester

Concentrated sulfuric acid (40 ml) was added to a stirred solution of 1-bromo-4-oxo-3,4-dihydro-phthalazine-6-carboxylic acid (85 g, 0.32 mol) in ethanol (500 ml) and the mixture was heated to reflux for 48 hours. After this time, the reaction mixture was cooled and the resulting precipitate was filtered. The precipitate was partitioned between ethyl acetate (1 L) and saturated NaHCO₃ (500 ml), the organic layer was separated and washed with water (500 ml) before being dried (MgSO₄), filtered and concentrated under vacuum to give the title compound (30 g, 31% yield) as a white solid. Tr=1.23 min, m/z (ES⁺) (M+H)⁺ 297 & 299

### 1-Bromo-3-isopropyl-4-oxo-3,4-dihydro-phthalazine-6-carboxylic acid ethyl ester

1-Bromo-4-oxo-3,4-dihydro-phthalazine-6-carboxylic acid ethyl ester (6 g, 0.02 mol) was dissolved in DMF (60 ml). To this was added NaH (60%, 0.97 g, 0.024 mol) as a DMF suspension (5 ml). The mixture was stirred at room temperature for 30 minutes then 2-bromo-propanol (3.7 g, 0.03 mol) was added in one portion as a solution in DMF (5 ml). The reaction mixture was stirred for 48 hours whereupon LC-MS showed complete consumption of starting material. The DMF was removed under vacuum and the resulting residue was partitioned between DCM (100 ml) and water (100 ml), the organic layer was dried (MgSO₄), filtered and concentrated under vacuum. The resulting yellow oil was recrystallised from methanol to give the title compound (2.3 g, 34% yield) as a white solid. Tr=1.75 min, m/z (ES⁺) (M+H)⁺ 339 & 341

### 4-Bromo-7-hydroxymethyl-2-isopropyl-2H-phthalazin-1-one

1-Bromo-3-isopropyl-4-oxo-3,4-dihydro-phthalazine-6-carboxylic acid ethyl ester (2.3 g, 6.8 mmol) was suspended in THF (50 ml) and cooled to 0° C. To the suspension was added LiBH₄ (5.1 ml of a 2M solution in THF, 10.2 mmol) dropwise, the suspension was allowed to warm to room temperature and stirred for 24 hours. After this time, LC-MS showed 50% starting material remained. To this was added LiBH₄ (1.7 ml of a 2M solution in THF, 3.4 mmol) and the reaction mixture was stirred for a further 3 hours. The reaction was cooled to 0° C., saturated NH₄Cl (40 ml) was added and the reaction mixture was then partitioned between water (50 ml) and DCM (150 ml). The organic layer was separated, dried (MgSO₄), filtered and concentrated under vacuum. The resulting residue was then purified by flash column chromatography (elution: 50% toluene, 30% ethyl acetate, 20% DCM) to give the title compound (0.9 g, 43% yield) as a white solid.

δ_{H} (400 MHz, DMSO), 8.28 (1H, s), 7.96 (1H, d), 7.88 (1H, d), 5.64 (1H, t), 5.31-5.18 (1H, m), 4.78 (2H, d), 1.35 (6H, d) Tr=1.31 min, m/z (ES⁺) (M+H)⁺ 297 & 299

### 4-Bromo-7-bromomethyl-2-isopropyl-2H-phthalazin-1-one

A solution of 4-bromo-7-hydroxymethyl-2-isopropyl-2H-phthalazin-1-one (0.74 g, 2.5 mmol) in acetonitrile (5 ml) was added dropwise to a stirred suspension of trimethylsilylbromide (TMSBr) (0.9 g, 6.3 mmol) and LiBr (0.41 g, 5 mmol) in acetonitrile (15 ml). The reaction mixture was heated to 80° C. for 24 hours, after which time the reaction mixture was cooled to room temperature and the solvent removed under vacuum. The resulting residue was purified by flash column chromatography (elution: 85% heptane, 15% ethyl acetate) to give the title compound (0.4 g, 44% yield) as a white solid.

δ_{H} (250 MHz, DMSO), 8.37 (1H, s), 8.03 (1H, d), 7.94 (1H, d), 5.26-5.09 (1H, m), 4.93 (2H, s), 1.35 (6H, d).

### 4-Bromo-7-cyclopropylmethoxymethyl-2-isopropyl-2H-phthalazin-1-one

Cyclopropylmethanol (0.05 ml, 0.67 mmol) was dissolved in THF (1 ml). To this was added NaH (60%, 0.028 g, 0.72 mmol) in a single portion. The mixture was stirred at room temperature for 5 minutes then 4-bromo-7-bromomethyl-2-isopropyl-2H-phthalazin-1-one (0.2 g, 0.56 mmol) in THF (1 ml) was added in one portion and the reaction mixture was stirred for 1 hour. Whereupon LC-MS indicated complete consumption of starting material, the solvent was removed under vacuum and the residue was purified by flash column chromatography (elution: 80% heptane, 20% ethyl acetate) to give the title compound (0.17 g, 87% yield) as a light yellow oil. Tr=1.80 min, m/z (ES⁺) (M+H)⁺ 351 & 353

This material was then used in the Buchwald reaction as described in Method A to give the corresponding 7-cyclopropylmethoxymethyl-2-isopropyl-4-(5-methyl-1H-pyrazol-3-ylamino)-2H-phthalazin-1-one (E-1).

### Example E-1

### 7-Cyclopropylmethoxymethyl-2-isopropyl-4-(5-methyl-1H-pyrazol-3-ylamino)-2H-phthalazin-1-one

δ_{H} (400 MHz, DMSO), 11.92 (1H, br s), 9.15 (1H, s), 8.42 (1H, d), 8.24 (1H, s), 7.80 (1H, d), 6.36 (1H, s), 5.29-5.21 (1H, m), 4.68 (2H, s), 2.25 (2H, s), 1.32 (6H, d), 1.12-1.03 (1H, m), 0.53-0.47 (2H, m) Tr=1.80 min, m/z (ES⁺) (M+H)⁺ 368.35.

### Example E-2

### 2-Isopropyl-4-(5-methyl-1H-pyrazol-3-ylamino)-7-(pyridin-3-ylmethoxymethyl)-2H-phthalazin-1-one

δ_{H} (400 MHz, DMSO), 11.92 (1H, s), 9.17 (1H, s), 8.55-8.53 (1H, m), 8.45 (1H, d), 8.31-8.29 (1H, m), 7.89-7.81 (2H, m), 7.52 (1H, d), 7.34-7.30 (1H, m), 6.37 (1H, s), 5.29-5.21 (1H, m), 4.83 (2H, s), 4.68 (2H, s), 2.25 (3H, s), 1.32 (6H, d) Tr=1.55 min, m/z (ES⁺) (M+H)⁺ 405.31.

### Example E-3

### 2-Isopropyl-4-(5-methyl-2H-pyrazol-3-ylamino)-7-(2-morpholin-4-yl-ethoxymethyl)-2H-phthalazin-1-one

δ_{H} (400 MHz, DMSO), 9.19 (1H, br s), 8.42 (1H, d), 8.23 (1H, s), 7.82 (1H, d), 6.34 (1H, s), 5.30-5.21 (1H, m), 4.69 (1H, s), 3.61 (2H, t), 3.58-3.54 (4H, m), 2.54 (2H, t), 2.44-2.39 (4H, m), 2.24 (3H, s), 1.32 (6H, s) Tr=1.52 min, m/z (ES⁺) (M+H)⁺ 427.38.

### Method F

### Example F-1

### 2-Isopropyl-4-(5-methyl-1H-pyrazol-3-ylamino)-7-methylsulfanylmethyl-2H-phthalazin-1-one

### 4-Bromo-2-isopropyl-7-methylsulfanylmethyl-2H-phthalazin-1-one

Sodium methanethiolate (0.23 g, 3.33 mmol) was dissolved in THF (1 ml) and added dropwise to a solution of 4-bromo-7-bromomethyl-2-isopropyl-2H-phthalazin-1-one (0.4 g, 1.11 mmol) in THF (10 ml). The mixture was stirred at room temperature for 3 hours whereupon LC-MS indicated complete consumption of starting material. The reaction mixture was diluted with water (20 ml) and extracted with ethyl acetate (50 ml). The organic layer was separated, dried (MgSO₄), filtered and concentrated under vacuum. The residue was subjected to flash column chromatography (elution: 80% heptane, 20% ethyl acetate) to give the title compound (0.29 g, 79% yield) as a white solid.

This material was then used in the Buchwald reaction as described in Method A to give the corresponding 2-isopropyl-4-(5-methyl-1H-pyrazol-3-ylamino)-7-methylsulfanylmethyl-2H-phthalazin-1-one (F-1).

### Example F-1

### 2-Isopropyl-4-(5-methyl-1H-pyrazol-3-ylamino)-7-methylsulfanylmethyl-2H-phthalazin-1-one

δ_{H} (400 MHz, DMSO), 9.15 (1H, s), 8.41 (1H, d), 8.20 (1H, d), 7.82 (1H, d), 6.36 (1H, s), 5.31-5.19 (1H, m), 3.90 (2H, s), 2.24 (3H, s), 1.95-1.93 (3H, m), 1.32 (6H, d) Tr=1.75 min, m/z (ES⁺) (M+H)⁺ 344.29.

### Example F-2

### 2-Isopropyl-7-methanesulfonylmethyl-4-(5-methyl-1H-pyrazol-3-ylamino)-2H-phthalazin-1-one

Oxone (0.43 g, 0.7 mmol) was added in one portion to a stirred solution of 2-isopropyl-4-(5-methyl-1H-pyrazol-3-ylamino)-7-methylsulfanylmethyl-2H-phthalazin-1-one (0.06 g, 0.17 mmol) in a 4:1 mixture of dioxane/water (1.2 ml) and the reaction mixture was stirred at room temperature for 1 hour. The reaction mixture was diluted with water (5 ml) and the solution was extracted with ethyl acetate (3×75 ml), the organic layers were combined, dried (MgSO₄), filtered and concentrated under vacuum to give the title compound (0.013 g, 20% yield) as a white solid.

δ_{H} (400 MHz, DMSO) 9.23 (1H, s), 8.48 (1H, d), 8.38 (1H, d), 7.89 (1H, dd), 6.36 (1H, s), 5.33-5.20 (1H, m), 4.77 (2H, s), 2.96 (3H, s), 2.25 (3H, s), 1.33 (6H, d) Tr=1.58 min, m/z (ES⁺) (M+H)⁺ 376.24.

### Method G

### Example G-1

### N-[3-Isopropyl-1-(5-methyl-1H-pyrazol-3-ylamino)-4-oxo-3,4-dihydro-phthalazin-6-yl]-2-methoxy-N-methyl-acetamide

### N-(1-Bromo-3-isopropyl-4-oxo-3,4-dihydro-phthalazin-6-yl)-2-methoxy-N-methyl-acetamide

4-Bromo-2-isopropyl-7-methylamino-2H-phthalazin-1-one (0.5 g, 1.69 mmol) was dissolved in DMF (5 ml). To this was added NaH (60%, 0.19 g, 5.1 mmol) as a suspension in DMF (2 ml). The mixture was stirred at room temperature for 30 minutes then methoxyacetyl chloride (0.27 g, 2.5 mmol) was added dropwise and the reaction mixture was stirred at room temperature for 24 hours. After this time, water (10 ml) was added cautiously, the mixture was extracted with ethyl acetate (3×10 ml), the organic layers were combined, dried (MgSO₄), filtered and concentrated under vacuum. Flash column chromatography (elution: 50% hexane, 50% ethyl acetate) gave the title compound (0.47 g, 76% yield) as a white solid.

This material was then used in the Buchwald reaction as described in Method A to give the corresponding 2-isopropyl-7-methylamino-4-(5-methyl-1H-pyrazol-3-ylamino)-2H-phthalazin-1-one.

2-Isopropyl-7-methylamino-4-(5-methyl-1H-pyrazol-3-ylamino)-2H-phthalazin-1-one (0.4 g, 1.3 mmol) was dissolved in DMF (5 ml). To this was added NaH (60%, 0.1 g, 2.6 mmol) as a suspension in DMF (2 ml). The mixture was stirred at room temperature for 5 minutes then methoxyacetyl chloride (0.28 g, 2.6 mmol) was added dropwise and the reaction mixture was stirred at room temperature for 2 hours. After this time, water (10 ml) was added cautiously, the mixture was extracted with ethyl acetate (3×10 ml), the organic layers were combined, dried (MgSO₄), filtered and concentrated under vacuum. The residue was dissolved in THF (5 ml) and heated to 60° C. in the presence of solid sodium hydroxide (100 mg) for 2 hours. After this time the mixture was concentrated under vacuum and subjected to flash column chromatography (elution: 95% ethyl acetate, 5% methanol) to give N-[3-isopropyl-1-(5-methyl-1H-pyrazol-3-ylamino)-4-oxo-3,4-dihydro-phthalazin-6-yl]-2-methoxy-N-methyl-acetamide (0.06 g, 12% yield) as a white solid.

### Example G-1

### N-[3-Isopropyl-1-(5-methyl-1H-pyrazol-3-ylamino)-4-oxo-3,4-dihydro-phthalazin-6-yl]-2-methoxy-N-methyl-acetamide

δ_{H} (400 MHz, DMSO), 9.25 (1H, s), 8.48 (1H, d), 8.17 (1H, d), 7.89 (1H, dd), 6.35 (1H, s), 5.32-5.17 (1H, m), 4.05-3.92 (2H, m), 3.28 (3H, s), 3.22 (3H, s), 2.25 (3H, s), 1.32 (6H, d) Tr=1.56 min, m/z (ES⁺) (M+H)⁺ 385.29.

### Example G-2

### N-[3-(3,5-Difluoro-benzyl)-1-(5-methyl-1H-pyrazol-3-ylamino)-4-oxo-3,4-dihydro-phthalazin-6-yl]-2-methoxy-N-methyl-acetamide

δ_{H} (400 MHz, DMSO), 9.32 (1H, br s), 8.49 (1H, d), 8.20 (1H, d), 7.92 (1H, dd), 7.22-7.02 (3H, m), 6.06 (1H, br s), 5.26 (2H, s), 4.01 (2H, br s), 3.29 (3H, s), 3.22 (3H, s), 2.17 (1H, s) Tr=1.80 min, m/z (ES⁺) (M+H)⁺ 468.95.

### Example G-3

### N-[3-Isopropyl-1-(5-methyl-1H-pyrazol-3-ylamino)-4-oxo-3,4-dihydro-phthalazin-6-yl]-N-methyl-2-phenoxy-acetamide

δ_{H} (400 MHz, DMSO), 9.41 (1H, s), 8.50 (1H, d), 8.26 (1H, s), 7.99 (1H, d), 7.31-7.13 (4H, m), 7.10-7.05 (1H, m), 6.91 (1H, t), 6.87-6.78 (2H, m), 6.08 (1H, s), 5.28 (2H, s), 4.78 (2H, br s), 2.19 (3H, s) Tr=2.07 min, m/z (ES⁺) (M+H)⁺ 530.97.

### Method H

### Example H-1

### 4-[3-Isopropyl-1-(5-methyl-1H-pyrazol-3-ylamino)-4-oxo-3,4-dihydro-phthalazin-6-ylamino]-butyric acid

### 4-Bromo-2-isopropyl-7-(2-oxo-pyrrolidin-1-yl)-2H-phthalazin-1-one

To a solution of 7-amino-2-isopropyl-4-bromo-2H-phthalazin-1-one (0.5 g, 1.8 mmol) in DMF (8 ml), was added triethylamine (0.28 ml, 1.98 mmol). After five minutes, 4-chlorobutyryl chloride (0.22 ml, 2.0 mmol) was added and the solution was stirred at room temperature for 2 hours. After this time LC-MS indicated the complete consumption of starting material and the mixture was diluted with DCM (30 ml) and washed with hydrochloric acid (1M, 20 ml). The organic layer was separated, dried (MgSO₄) and concentrated under vacuum. The residue was subjected to flash column chromatography (elution: 60% heptane, 40% ethyl acetate) to give N-(1-bromo-3-isopropyl-4-oxo-3,4-dihydro-phthalazin-6-yl)-4-chloro-butyramide (0.46 g, 67% yield) as a white solid.

This material was dissolved in DMF (5 ml). To this was added NaH (60%, 0.05 g, 1.3 mmol) as a suspension in DMF (2 ml). The mixture was stirred at room temperature for 2 hours. After this time, water (10 ml) was added cautiously, the mixture was extracted with ethyl acetate (3×10 ml) and the organic layers were combined, dried (MgSO₄), filtered and concentrated under vacuum. The residue was subjected to flash column chromatography (elution: 60% heptane, 40% ethyl acetate) to give the title compound (0.15 g, 36% yield) as a pale yellow solid.

This material was then used in the Buchwald reaction as described in Method A. to give the corresponding 4-[3-Isopropyl-1-(5-methyl-1H-pyrazol-3-ylamino)-4-oxo-3,4-dihydro-phthalazin-6-ylamino]-butyric acid (H-1)

### Example H-1

### 4-[3-Isopropyl-1-(5-methyl-1H-pyrazol-3-ylamino)-4-oxo-3,4-dihydro-phthalazin-6-ylamino]-butyric acid

δ_{H} (400 MHz, DMSO) 8.10 (1H, s), 7.08 (1H, d), 7.02-6.83 (2H, m), 6.16 (1H, s), 5.27-5.01 (1H, m), 3.02 (2H, d), 2.08 (3H, s), 1.99 (2H, t), 1.78-1.54 (2H, m), 1.19 (6H, d) Tr=1.60 min, m/z (ES⁺) (M+H)⁺ 385.43.

1. A compound of formula I, 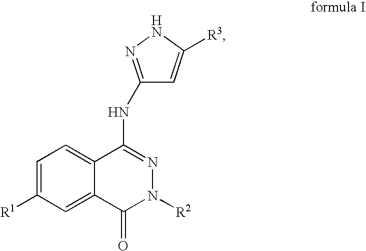
wherein
R¹ is R⁴—X— or
R⁵—X-alkylene-;
R⁴ is alkyl wherein said alkyl is substituted one to three times by hydroxy, alkoxy, carboxy, amino, alkylamino, dialkylamino, alkylsulfanyl, alkylsulfinyl, alkylsulfonyl, alkylsulfamoyl, dialkylsulfamoyl, alkylsulfonylamino, phenoxy or heterocyclylsulfonyl;
R⁵ is selected from the group consisting of:
alkyl,
cycloalkyl-T-,
heterocyclyl-T-,
aryl-T-, and
heteroaryl-T-;
X is selected from the group consisting of: —S(O)₂—, —S(O)—, —C(O)NR—, —NR—, —O—, and —S—;
T is a single bond or alkylene;
R² is alkyl, or
arylalkyl, wherein the aryl is substituted one to three times by halogen;
R³ is selected from the group consisting of: hydrogen, alkyl, and cycloalkyl;
R is hydrogen or alkyl;
or a pharmaceutically acceptable salt thereof.

2. A compound according to claim 1, wherein
R⁴ is alkyl wherein said alkyl is substituted one or two times by alkoxy, carboxy, dialkylamino, alkylsulfanyl, alkylsulfinyl, alkylsulfonyl, dialkylsulfamoyl, phenoxy or heterocyclylsulfonyl;
R⁵ is selected from the group consisting of:
alkyl,
cycloalkyl-T-,
heterocyclyl-T-, and
heteroaryl-T-;
X is selected from the group consisting of: —S(O)₂—, —C(O)NR—, —NR—, —O—, and —S—; and
R³ is alkyl.

3. A compound according to claim 1, wherein
R² is alkyl.

4. A compound according to claim 1, wherein
R² is arylalkyl, wherein the aryl is substituted one to three times by halogen.

5. A compound according claim 1 selected from the group consisting of:
2-Isopropyl-4-(5-methyl-1H-pyrazol-3-ylamino)-7-(2-methylsulfanyl-ethoxy)-2H-phthalazin-1-one;
2-Isopropyl-7-(2-methoxy-ethoxy)-4-(5-methyl-1H-pyrazol-3-ylamino)-2H-phthalazin-1-one;
2-Isopropyl-7-(2-methanesulfonyl-ethoxy)-4-(5-methyl-1H-pyrazol-3-ylamino)-2H-phthalazin-1-one;
2-Isopropyl-7-[methyl-(2-methylsulfanyl-ethyl)-amino]-4-(5-methyl-1H-pyrazol-3-ylamino)-2H-phtalazin-1-one;
4-(4-Bromo-5-methyl-1H-pyrazol-3-ylamino)-2-isopropyl-7-(2-methylsulfanyl-ethoxy)-2H-phthalazin-1-one;
2-Isopropyl-7-[(2-methoxy-ethyl)-methyl-amino]-4-(5-methyl-1H-pyrazol-3-ylamino)-2H-phthalazin-1-one;
4-(4-Bromo-5-methyl-1H-pyrazol-3-ylamino)-2-isopropyl-7-(2-methanesulfinyl-ethoxy)-2H-phthalazin-1-one;
2-Isopropyl-7-(2-methane sulfinyl-ethoxy)-4-(5-methyl-1H-pyrazol-3-ylamino)-2H-phthalazin-1-one;
2-Isopropyl-4-(5-methyl-1H-pyrazol-3-ylamino)-7-(2-methylsulfanyl-ethylsulfanyl)-2H-phthalazin-1-one;
7-[(2-Dimethylamino-ethyl)-methyl-amino]-2-isopropyl-4-(5-methyl-1H-pyrazol-3-ylamino)-2H-phthalazin-1-one;
3-[3-Isopropyl-1-(5-methyl-1H-pyrazol-3-ylamino)-4-oxo-3,4-dihydro-phthalazin-6-yloxy]-propane-1-sulfonic acid dimethylamide;
2-Isopropyl-4-(5-methyl-1H-pyrazol-3-ylamino)-7-[3-(morpholine-4-sulfonyl)-propoxy]-2H-phthalazin-1-one;
2-Isopropyl-7-(2-methanesulfonyl-ethanesulfonyl)-4-(5-methyl-1H-pyrazol-3-ylamino)-2H-phthalazin-1-one;
7-(2-Dimethylamino-ethoxy)-2-isopropyl-4-(5-methyl-1H-pyrazol-3-ylamino)-2H-phthalazin-1-one;
7-(2-Dimethylamino-ethylamino)-2-isopropyl-4-(5-methyl-1H-pyrazol-3-ylamino)-2H-phthalazin-1-one;
4-[3-Isopropyl-1-(5-methyl-1H-pyrazol-3-ylamino)-4-oxo-3,4-dihydro-phthalazin-6-ylamino]-butyric acid;
N-[3-Isopropyl-1-(5-methyl-1H-pyrazol-3-ylamino)-4-oxo-3,4-dihydro-phthalazin-6-yl]-2-methoxy-N-methyl-acetamide;
N-[3-(3,5-Difluoro-benzyl)-1-(5-methyl-1H-pyrazol-3-ylamino)-4-oxo-3,4-dihydro-phthalazin-6-yl]-2-methoxy-N-methyl-acetamide; and
N-[3-Isopropyl-1-(5-methyl-1H-pyrazol-3-ylamino)-4-oxo-3,4-dihydro-phthalazin-6-yl]-N-methyl-2-phenoxy-acetamide;
7-Cyclopropylmethoxymethyl-2-isopropyl-4-(5-methyl-1H-pyrazol-3-ylamino)-2H-phthalazin-1-one;
2-Isopropyl-4-(5-methyl-1H-pyrazol-3-ylamino)-7-(pyridin-3-ylmethoxymethyl)-2H-phthalazin-1-one;
2-Isopropyl-4-(5-methyl-2H-pyrazol-3-ylamino)-7-(2-morpholin-4-yl-ethoxymethyl)-2H-phthalazin-1-one;
2-Isopropyl-4-(5-methyl-1H-pyrazol-3-ylamino)-7-methylsulfanylmethyl-2H-phthalazin-1-one; and
2-Isopropyl-7-methanesulfonylmethyl-4-(5-methyl-1H-pyrazol-3-ylamino)-2H-phthalazin-1-one.

6. (canceled)

7. A pharmaceutical composition, comprising a compound according to claim 1 and a pharmaceutically-acceptable excipient.

8-10. (canceled)
